(19) United States
(12) Patent Application Publication
(10) Pub. No.: US 2016/0266141 A1
(43) Pub. Date: Sep. 15, 2016

(54) MASS SPECTROMETRY-BASED METHOD FOR IDENTIFYING AND MAINTAINING QUALITY CONTROL FACTORS DURING THE DEVELOPMENT AND MANUFACTURE OF A BIOLOGIC

(71) Applicant: BIOANALYTIX, INC., Cambridge, MA (US)

(72) Inventors: Lily T. Li, Lexington, MA (US); Kirtland G. Poss, Marblehead, MA (US); Robert Garnick, Las Vegas, NV (US); Shiaw-Lin Wu, Weston, MA (US)

(73) Assignee: BIOANALYTIX, INC., Cambridge, MA (US)

(21) Appl. No.: 15/026,514

(22) PCT Filed: Oct. 3, 2014
(86) PCT No.: PCT/US2014/059150

§ 371 Date: Mar. 31, 2016

Related U.S. Application Data
(60) Provisional application No. 61/886,208, filed on Oct. 3, 2013.

(51) Int. Cl. G01N 33/68

(52) U.S. Cl. CPC G01N 33/6848

(57) ABSTRACT

The invention provides methods for determining a set of critical features that serves as a “map” of attributes critical for maintaining the structure and function of a biologic. The map can serve as a development tool, e.g., as a guide or target during the development of expression, purification, and formulation protocols, a quality assurance tool during manufacturing, or as a definitive identifier of the specific biologic. The map can also serve as the definition of the biologic thereby providing a means by which a given product may be reliably characterized as a biosimilar of another biologic product.

### CROSS-REFERENCE TO RELATED APPLICATIONS

This application claims the benefit of and priority to U.S. Provisional Patent Application No. 61/886,208, filed Oct. 3, 2013, the entire disclosure of which is incorporated by reference herein in its entirety.

### FIELD OF THE INVENTION

The invention relates generally to the development and control of manufacture of biological molecules (biologics or biological drugs), such as protein and peptide drugs, derived from biologic tissue or made by exploiting recombinant DNA techniques. More particularly, the invention relates to a process for definitively characterizing biologics and their key attributes, and to methods of determining a set of related process parameters critical to successful and reproducible development, manufacture, formulation, storage, and administration of a biologic that assures consistent achievement and maintenance of its safety, purity, potency and efficacy.

### BACKGROUND

Experience with biological drugs over the past decades has shown that their development and subsequent manufacture and approvability are intimately related. Development of a new biologic (also referred to herein as a biological drug) can be viewed as an enormous combinatorial puzzle where many process steps and materials affect many others, and where even small changes in process can lead to variations in the resulting biologic's structure, stability and resulting function. Some process steps and materials are absolutely critical to achieving product specifications consistently, others less so. The puzzle typically is solved empirically by trial and error, where one batch of drug is tested in vivo and/or clinically to assess related efficacy. A limitation in the current art is in determining and characterizing what structural attributes of a biologic drug molecule actually matter to its desired stability and function, and how to produce batches of drug that consistently have the same physical and chemical properties and, therefore, the same stability plus efficacy/toxicity profile. In cases where the problem is solved, the solution usually becomes a closely guarded trade secret.

Biologics are highly complex molecules that can be expressed and refolded incorrectly based on a range of variations in the biosynthesis or manufacturing process, and can be degraded or chemically changed by proteases, heat, acidic or other environmental conditions to produce fragments and truncated molecules. Some biologics tend to form aggregates, which are inactive and sometimes immunogenic. Biologics can be glycosylated at differing N-linked or O-linked sites, by different amounts, and/or with different sugars (e.g., they may vary by galactose content, afucosylation, sialic acid content, mannose content, etc.), and may include molecular species non-glycosylated at critical or uncritical locations. Proper disulfide bonding within a molecule and/or between molecules typically is critical for efficacy, and wrongly paired or unpaired disulfide bonds can lead to inoperative misfolded contaminants. The product also may be contaminated by one or more host cell proteins such as proteases, DNAs, methotrexate, or other residues from upstream expression, or with leached components from downstream purification. They also may be deaminated, oxidized, methylated or otherwise derivatized. The molecule may be altered after its release, in storage, or in vivo when exposed to blood-borne enzymes, physiological temperatures, and the like.

Production methods profoundly affect structure in various ways. A master cell bank comprising replicable, recombinant clones that reliably express copious quantities of active biologic is only a beginning. Upstream variables in culture of such cells such as culture duration, pH, amount of dissolved oxygen, concentrations and identities of media components, temperature, initial cell density, pCO₂, mixing and gassing strategy, and feeding strategy each may affect not only the quantitative protein yield, but also the structure of the product. Furthermore, contaminants such as host cell proteins, metabolites and the like are inevitably introduced into extracellular broths, as are possible infective agents such as viruses. Similarly, the downstream purification process may introduce variants or contaminants that may alter protein structure. The fine structure of the product can be affected by such aspects as the selection of separation technologies such as affinity columns, anionic or cationic exchange columns, or ultrafiltration apparatus. Also, contaminants may be introduced or product degraded or derivatized by the addition of preservatives, diluents, vehicle, as well as the decision as to when a chromatography resin or filter is replaced, and the temperature or pH of the product during purification, compounding, and storage.

Heretofore, developers of biologics have determined empirically what resulting structure works, how to reproducibly manufacture that structure, and then postulate release criteria dictated by the necessity of achieving reproducible product meeting all regulatory requirements. Often, the fine structure of the molecule is unknown, and the key attributes of the molecule that determine aspects such as final stability or efficacy are also unknown. A change in an expression or purification parameter, or a change in a media component or other consumable used in manufacture, can have a subtle but important and unpredictable effect on the product, even while the mechanism of such change remains unknown. Sometimes, the structural change induced is subtle and makes no detectable difference in safety, purity, or potency. Sometimes a change affects efficacy because of a change in the structure of the molecule, which occurs only in vivo. Developers and manufactures of innovator biologic drugs often must try to replicate the resulting stability and efficacy from batch to batch of drug. Manufacturers of biosimilars, by definition not privy to details of the originator's CMC protocols, must try to replicate a molecule whose fine structure is profoundly affected by its method of manufacture, and that method is unknown. Furthermore, the precise structure of the biologic drug they seek to replicate, e.g., the relevant pattern of glycosylation or of disulfide bridges, often also is unknown.

The established generic approach for producing small molecule drugs, involving precise chemical replication and demonstration of biological equivalence, is clearly scientifically insufficient for far more complex biological/biotechnology derived products. Recently, mass spectrometry-based methods have been developed for characterizing degradation products and chemical modifications of biologics. However, there is still an ongoing need for a process for definitively characterizing the key structures of a biologic drug candidate and for a method of determining the related set of process parameters critical to successful, reproducible development, manufacture, formulation, storage and administration of a biologic that assures consistent achievement and maintenance of its safety, efficacy and potency.

### SUMMARY

The inventors have developed an approach to enable developers and manufacturers of biologics or biosimilars to better control and maintain biologic structure, in particular, the key biological structures related to stability and efficacy, of a biologic molecule over the entire interval of its existence from its synthesis to its consumption by a patient. The approach involves the creation of a map (or fingerprint) that comprises a set of key parameters which characterize important aspects of the fine structure, and key structures, of a given biologic molecule, and link the structure(s) to the aspects of its development, manufacture, formulation and storage that create and maintain that structure. The map can serve as a quality assurance tool that enables consistent reproducibility of the key structures and related properties of a given biologic. It also can provide criteria for development and approval as a biosimilar. A properly derived set of analytic criteria for confirming the relevant structural features of proteins known to be important for effectiveness (or not), and derived through exploration of the links between those critical structural features, their effect on stability and biological activity, and to process parameters, can be a key driver in successful development of both biologics and biosimilars. Such analytical, chemical and biological characterization of key features will be important in demonstrating that a proposed biologic or biosimilar product qualifies as “highly similar” to a reference batch or product.

Accordingly, the invention provides a systematic process that also can be provided as a service, for creating a map of key features in the structure of a biologic that are critical to its stability, safety, purity, efficacy, and/or potency. This map identifies, among other things, what features of the biologic are critical for efficacy, what substructures are susceptible to degradation or modification, and what areas may induce aggregation. It directly informs development of reproducible manufacture and control protocols, and permits the prediction of the effect of selection of particular starting materials or manufacturing process steps. Such a map also provides developers, manufacturers, and potentially regulators, with both prospective and retrospective assurances that product quality specifications will be and have been met, and permits the development and production of consistent quality product. The map also can inform development of in-process testing, release testing, process monitoring, characterization and/or comparability testing, and regulatory program criteria and design relating to both the most relevant and least relevant structural features of the drug.

In accordance with the invention, there has now been devised an efficient and sensitive, generally applicable method for developing such a tool for any given biologic. The method is based on inter-related mass spectrometry and bioassay testing, and ultimately results in the creation of a “map” that uniquely characterizes a given biologic and/or describes substructures of the molecule subject to material change, i.e., changes that affect safety, purity, potency and/or efficacy (“labile substructures”), as a result of exposure to conditions of manufacture, storage, or formulation. The map optionally also identifies, directly or by inference, processing, storage or formulation conditions sufficient to maintain the fine structure and drug properties of the molecule. The biologic map is produced by the combined execution of protein preparation, mass spectrometry-based structural analyses and cell or process based stability and biological function testing such as efficacy.

In a first phase, various features of the fine structure of the biologic are determined. The biologic is known (or expected, based for example on data generated through at least pre-clinical studies) to exhibit a desired biological activity thought to support some prophylactic or therapeutic utility, i.e., it comprises at least a lead compound. This phase of the process typically operates on a reference biologic. By exploiting recently developed analysis techniques, the invention contemplates as one step characterizing the primary, secondary, tertiary and quaternary structure of the molecule. It determines such things as the precise pattern of post-translational modifications, maps intra and inter-chain disulfide bonds, including the presence of any disulfide knots, determines whether and where the molecule is glycosylated, the nature of the glycosylation, whether the molecule is truncated after expression, whether its activity is dependent on covalent or non-covalent dimerization, and generally, the nature of its post-translational modification(s). This phase of the analysis is essentially an application of recently developed and continually improving mass spectrometry-based analysis techniques and is used to generate (through mass spectrometry analysis) data indicative of the fine structure of an active, reference biologic.

In a second phase, the reference biologic is subjected to various conditions or tests that (a) stress the molecule potentially to result in its modification, degradation, denaturation, contamination, instability or aggregation, and/or (b) assess the efficacy/safety of the reference biologic including in cell-based, in vivo or clinical assays in order to determine overall stability and/or efficacy/safety profiles. Thus, for example, the reference biologic is subjected to high temperature, physiological temperature, light, pH changes, enzymes that are commonly present in production broths, lyophilization and reconstitution, changes in ionic environment, mechanical stresses such as filtration and other separation techniques, accelerated aging conditions, and/or conditions that the molecule might encounter in vivo, such as physiological temperature, and various biomolecules (e.g., proteases), ions, or dissolved gases.

The as-stressed reference biologic, and/or its derivatives, fragments, or degradation products are themselves analyzed, for example, to determine the effect, if any, of the stress on its structure. The structures then are compared to that of the intact, reference biologic, and optionally, as may be necessary, activity assays are conducted on the intact and/or as-stressed molecular species which exhibit an alteration in structure. This process results in (for example, by mass spectrometric analysis) data indicative of the structures of the as-stressed reference biologic, and optionally derivatives, fragments, or degradation products thereof. The data then can be analyzed computationally to determine which operational parameters used in the expression, purification, formulation or storage of the reference biologic result in or pose a risk of degrading, modifying, or contaminating the biological drug.

Thereafter, these data are used to create a record of structural features of the molecule, that is, a map which identifies and characterizes parts of the molecular structure at risk of modification when exposed to various conditions. Furthermore, the map can reveal which particular attributes or modifications affect the molecule's stability or activity, which attributes or modifications are innocuous, and what specific conditions induce the modifications. The map thus enables determination of (a) which attributes actually relate to and/or impart function, or not, and (b) which processing parameters degrade or risk degrading the structural features of the biologic known to be material to its function and safety. Stated differently, the map enables determination of which structural features of the protein matter and which don't, which features are susceptible to alteration or degradation caused by selection of particular raw materials used and/or processing steps employed in its expression, purification, or formulation, and from this directly informs downstream decisions in development, regulatory manufacturing, and Quality Assurance (QA)/Quality Control (QC) processes.

The map comprises a record of selected analyses and results thereof informative of the structural aspects of the drug that are critical to its stability and biologic activity, and specifies the conditions of expression, purification, formulation or storage to maximize the chance of producing safe and efficacious drug meeting all structural aspects that are critical to its stability and biological activity.

The map has several valuable utilities. It comprises a listing of a set of sites in the molecule as related to reproducible analytical techniques and their outputs which uniquely characterize the molecule. It may be exploited during development of expression and purification protocols conforming to GMP. It predicts triggers of, and development solutions to, degradation, aggregation, and aberrant modification. It can function as a quality control tool in commercial scale production of the biologic so as to ensure reliable manufacture of consistent quality product falling within realistic specifications guaranteeing its safety, purity, potency and efficacy. When, for whatever reason, a process step is changed before or after approval, the map enables comparability testing with variances in results computationally or statistically measured against the original map. It also serves as a specification or benchmark for long term stability testing, and importantly can serve as a roadmap for specific development and regulatory process designs and decisions, including tailored formulation to mitigate or enhance the identified key fine structures of the molecule.

Still another important utility is to serve as a standard in the development of biosimilars. Accordingly, in another aspect the invention contemplates analyzing an approved biologic drug using the foregoing techniques to develop a biological map, and then expressing and purifying a candidate biosimilar using various different raw materials, purification techniques and reagents until a biosimilar is produced which has a map that replicates the map of the previously approved biologic.

### BRIEF DESCRIPTION OF THE DRAWINGS

FIG. 1 is a flow chart showing different approaches, namely, bottom-up (Fragmentation¹: produced by enzymatic digestion using multiple enzymes), middle down (Fragmentation²: produced by enzymatic digestion using a single enzyme or chemical), or top-down approaches (Fragmentation³: produced via gas phase cleavage using collision induced dissociation (CID) and electron transfer dissociation (ETD)), for characterizing the primary sequence of a biologic, for example, a protein.

FIG. 2 is a schematic representation of a staggered strategy to improve the coverage of protein sequences using bottom-up (small peptides, blue fragments; left-most two protein representations), middle-down (large peptides, orange fragments; right-most two representations), and top-down approaches (intact protein, green fragments; middle representation).

FIG. 3 is an example of common peptide fragmentation ions produced by mass spectrometry, where (a) and (x) ions are produced by collision induced dissociation (CID) (blue dot lines; labeled a₁/x₃, a₂/x₂, and a₃/x₁); (b) and (y) ions are produced predominantly by collision induced dissociation (CID) (brown dot lines; labeled b₁/y₃, b₂/y₂, and b₃/y₁); (c) and (z) ions are produced predominantly by electron transfer dissociation (ETD) (green dot lines; labeled c₁/z₃, c₂/z₂, and c₃/z₁).

FIG. 4 is a schematic representation showing an approach for analyzing a protein containing disulfide bonds. Polypeptides P1 and P2 are connected through two inter-chain disulfide bonds denoted (2) and (3). The protein without reduction is digested with enzymes to produce disulfide-free peptides and three disulfide-linking peptides DSLP(1), DSLP(2), and DSLP(3). DSLPs then are analyzed by ETD-MS² and CID-MS³, and disulfide free peptides are fragmented by CID-MS² and CID-MS³.

FIG. 5 is a schematic representation of an exemplary process for characterizing a disulfide knot containing protein. A protein containing a disulfide knot is treated with a single enzyme (Enzyme A) and multiple enzymes, for example, Enzymes (B, C), (B, C, D), and/or (B, C, D, E) with no reduction and/or partial reduction. Disulfide free peptides and disulfide knot containing peptides (DSKCPs) then are analyzed using CID (CID-MS²), ETD (ETD-MS²), and CRCID (CID-MS³).

FIG. 6 is a schematic representation of an exemplary process for characterizing an N-linked glycosylated protein. Process 1 is a deglycosylation process using PNGase F to free N-linked glycans from glycoprotein (Step 1). The N-linked glycans (A) are separated from an intact protein (B) using molecular weight cut-off filters (Step 2). The isolated glycans (A) are subjected to glycan analysis (Step 3). Process 2 using multiple enzymes including PNGase F (Step 1) generates nonglycosylated and deglycosylated peptides (C) to be used for peptide sequencing by CID-MS² and HCD-MS² (Step 3). Process 3, Step 1 uses multiple enzymes without PNGase F to produce N-linked glycosylated and nonglycosylated peptides (D). N-linked glycosylated peptide sequencing is performed using ETD-MS², CRCID (CID-MS³), CID-MS², and/or HCD-MS² in Step 4. Use of CID-MS² and HCD-MS² on N-glycosylated peptides determines site-specific N-glycans in Step 5. Non-glycosylated peptide sequencing is carried out using CID-MS² and/or HCD-MS² in Step 6. N-linked glycosylation sites are confirmed as results of peptide mapping in Process 3/Step 2 and Step 4 as well as Process 2/Step 2 and Step 3.

FIG. 7 is a schematic representation of an exemplary process for characterizing an O-linked glycosylated protein. Process 1 employs multiple enzymes including PNGase F to produce non-glycosylated and O-linked glycosylated peptides in Step 1. O-linked glycosylated and non-glycosylated peptides then are monitored in Step 2 and Step 3, respectively, based on their predicted masses. O-glycosylated peptides are sequenced by ETD-MS², CRCID (CID-MS³), CID-MS², and/or HCD-MS² (Process 1/Step 5). The site-specific 0-glycans is determined using CID-MS² and/or HCD-MS² (Process 1/Step 4). The non-glycosylated peptide sequencing is carried out using CID-MS² and/or HCD-MS² in Step 6. As results of Steps 5 and 6 in Process 1, O-linked glycosylation sites are determined. Process 2 is deployed for glycan analysis. The Step 1 in Process 2 uses chemicals to release all glycans from a glycoprotein. Released glycans are isolated from an intact protein using molecular weight cut-off filters (Process 2, Step 2). Collected glycans are subjected to glycan analysis (Process 2/Step 3).

FIG. 8 is a schematic representation of an exemplary glycan analysis. Quantitative measurement is achieved by LC-FD-MS by adding fluorescent dye onto the reducing end of glycans (Derivatized Glycans¹). Various derivation methods are conducted as appropriate (Derivatized Glycans²). CID (CID-MS², CID-MS³, and possibly CID-MSⁿ) and HCD (HCD-MS²) are applied to glycan molecules (underivatized and/or derivatized) to characterize structures.

FIG. 9 is an exemplary profile of common fragment ions produced by mass spectrometry. CID and HCD can break glycosidic bonds to generate B, C, Y, and Z ions (blue dotted lines) as well as cross-rings to produce A and X ions (red dashed lines). D ions can be produced by breaking two glycosidic bonds (B and Y) at the branch (green dotted/dashed lines).

FIG. 10 is a schematic representation of a template for a map of a reference biologic constructed in accordance with the invention.

FIGS. 11A-11F are an exemplary structural characterization process of an exemplary monoclonal antibody (mAb, anti-CD20). FIG. 11A is a schematic representation of the characterization process. An intact mAb is measured by LC/MS, showing MWs containing heterogeneous glycan species in FIG. 11B. Digested peptides with reduction are observed in FIG. 11C (see the top panel for the LC/MS chromatogram of digested mAb). CID and ETD are carried out on each peptide fragment to confirm the sequence (see FIG. 11C, bottom panel for sequencing Pyro-Glu N-terminal). Through peptide mapping, N-glycosylation sites are determined using an mAb digest without PNGase F (FIG. 11D). The N-site specific glycoforms are identified using CID on N-glycopeptides (FIG. 11E). FIG. 11F illustrates the determination of disulfide bond location using an mAb digest without reduction (the top panel representing CID-MS² sequencing of a disulfide-linking peptide; the bottom panel representing ETD-MS² sequencing of a disulfide-linking peptide).

FIG. 12 is a schematic representation of using a CQA map for a comparability study of a biologic (for example, mAb) during manufacture. The comparability testing covers the intact MW measurement (see an example in FIG. 11B), peptide mapping and sequencing (see an example in FIG. 11C), determination of glycosylation site(s) (see an example in FIG. 11D), identification of glycoforms (see an example in FIG. 11E), location of disulfide bonds (see an example in FIG. 11F).

Other aspects, embodiments, and features will be become apparent from the following detailed description when considered in conjunction with the accompanying figures.

### DETAILED DESCRIPTION

There has been a need for a systematic process for mapping features of the precise structures of biological drugs that are critical to its safety, efficacy and/or potency, and for correlating creation and maintenance of these critical structural features to the physical and chemical conditions to which the molecule may be subjected to from the time of its synthesis to its administration to a patient. A detailed map of the structure of the biologic, complete with, for example, identification of which features of the molecule are critical for efficacy, what substructures are susceptible to degradation or modification, and what areas may induce aggregation, provides a tool that enables more effective and accurate development of reproducible manufacture and control protocols, and enables the prediction of the effect of selection of particular starting materials or manufacturing process steps.

The invention described herein therefore provides methods for determining a set of molecular structural features or attributes of a biologic drug that: a) must be maintained to assure consistent efficacy, safety, purity, and/or potency of a biologic, b) are potentially subject to alteration or degradation when and if the biologic is exposed to certain physical and chemical conditions, c) can be computationally or statistically evaluated in comparability testing of similar molecules and d) are maintained if certain conditions of manufacture, formulation, or storage are applied or maintained. Practice of the process results in a compilation of information that may serves as a map or fingerprint of molecular features and process conditions critical to maintain or reproduce the structure and function of the biologic. The map can serve as a development tool, e.g., as a guide or target during the development of expression, purification, and formulation protocols, after development as a quality assurance tool (akin to a detailed set of specifications that must be achieved during manufacturing), or as a definitive identifier of the specific biologic. It is contemplated that the map may serve as the definition of the biologic and provides the means by which a given product may be reliably reproduced and/or characterized as a biosimilar of another biologic product.

In one aspect, the invention relates to a method of generating a biologic map comprising data uniquely characterizing a biological drug exhibiting a desired biological activity. The method comprises (a) generating through mass spectroscopic analysis data indicative of the structure, for example, the fine structure, of an active, reference biologic; (b) subjecting the reference biologic to one or more stress conditions selected from high temperature, physiological temperature, pH change, light, lyophilization, reconstitution, changes in ionic environment, mechanical stress, and accelerated aging; (c) generating through mass spectroscopic analysis data indicative of the structure, for example, the fine structure, of the as-stressed reference biologic, and optionally derivatives, fragments or degradation products thereof; (d) computationally analyzing the data generated in step (a) and/or (c) to determine which operational parameters used in the expression, purification, formulation, or storage of said reference biologic result in or pose a risk of degrading, modifying, or contaminating the biological drug; and (e) preparing a map comprising a record of selected analyses and results thereof informative of the structural aspects of the reference biologic that are critical to its stability and biologic activity using the data generated in step (a) and/or (c), and optionally specifies the conditions of expression, purification, formulation, or storage maximizing the chance of producing safe and efficacious biologic drug meeting said structural aspects that are critical to its stability and biological activity.

As used herein, the terms “biologic” and “biological drug” relate to a drug or drug candidate that is produced by recombinant DNA technologies, peptide synthesis, or purified from natural sources and that has a desired biological activity. The biologic or biological drug can be, for example, a protein, peptide, glycoprotein, polysaccharide, a mixture of proteins or peptides, a mixture of glycoproteins, a mixture of polysaccharides, a mixture of one or more of a protein, peptide, glycoprotein or polysaccharide, or a derivatized form of any of the foregoing entities. The molecular weight of biologics can vary widely, from about 1000 Da for small peptides such as peptide hormones to one thousand kDa or more for complex polysaccharides, mucins, and other heavily glycosylated proteins. The biologic subject of the process of this invention can have a molecular weight of 1 kDa to 1000 kDa, more typically 20 kDa to 200 kDa, and often 30 kDa to 150 kDa. By way of example, desmopressin, oxytocin, angiotensin and bradykinin each have a molecular weight of about 1 kDa, calcitonion is 3.5 kDa, insulin is 5.8 kDa, kineret is 17.3 kDa, erythropoietin is about 30 kDa, ontak is 58 kDa, orencia is 92 kDa, and antibodies are approximately 150 kDa (Rituxan 145 kDa, Erbitux 152 kDa). Hyaluronic acids and salts have an average molecular weight often greater than 1000 kDa. As used herein, the term “reference biologic” refers to a biologic that is representative of the biological drug under development or that that has been approved for marketing, and provides a reference standard for the biological drug with, for example, the appropriate, predetermined composition, purity and/or biological activity.

In certain embodiments, the method further comprises the step of assaying the as-stressed reference biologic, and optionally derivatives, fragments or degradation products thereof for drug activity before step (e).

In certain embodiments, step (c) comprises determining the mass spectrometry profile in a sample of one or more stressed species selected from the group consisting of derivatized, truncated, oxidized, methylated, deaminated, aggregated, differentially glycosylated, improperly disulfide bonded, or structurally intact protein species, fragments thereof, and contaminants therein. Furthermore, the mass spectrometry analysis data generated in step (a) or step (c) is generated by fragmenting or chemically modifying the reference biologic, the as-stressed reference biologic, and optionally derivatives, fragments or degradation products therein before or while subjecting the sample to mass spectrometric analysis.

In certain embodiments, the generation of mass spectrometry data is effected through one or more of the techniques selected from the group consisting of electron transfer dissociation mass spectrometry, collision induced dissociation mass spectrometry, higher-energy collisional dissociation mass spectrometry, electron capture dissociation mass spectrometry, infrared multi-photon dissociation mass spectrometry, hydrogen/deuterium exchange mass spectrometry, MS², MS³, and LC-MS. Furthermore, the mass spectrometry analysis data in steps (a) or (c) can also be generated by analytical techniques selected from the group consisting of electrophoresis, selective proteolysis, UV spectra analysis, Fluorescent spectra analysis, IR spectra analysis and MRI spectra analysis.

In certain embodiments, the map additionally specifies the physical or chemical conditions which risk alteration of structural features of the biologic drug critical to its safety, purity, potency and efficacy. If appropriate, the map can be of sufficient detail to serve as quality assurance criteria to qualify a batch of the biological drug at some stage of its manufacture as biosimilar to another batch of the biological drug at the same stage produced separately. Similarly, the map can have sufficient detail to serve as criteria to qualify for regulatory purposes one batch of the biological drug as biosimilar to another batch of the biological drug produced separately. Furthermore, the map can be of sufficient detail to serve as criteria to qualify one batch of the biological drug as biosimilar to another batch of the biological drug produced separately using an altered protocol.

In another aspect, the invention provides a method of qualifying a given batch of biologic as biosimilar to a previously marketed biologic approved by a regulatory agency, the batch having been made by purification of expression products of a host cell in culture. The method comprising analyzing the batch to assure that its attributes satisfy a critical quality attribute map produced in accordance with the method described above. Once qualified, the batch can be marketed as an approved drug. In addition, another batch produced using the same protocol used to produce said given batch can be marked as an approved drug.

The method generally occurs in multiple phases that can occur simultaneously or sequentially. During a first phase, the biologic of interest is initially characterized. In a second phase, the biologic is interrogated to determine which of its features are material to its safety, purity, potency and/or efficacy, and how these features may be affected by exposure to physical or chemical conditions encountered by the biologic during expression, purification, formulation or storage. During a third phase, the resulting information can be compiled to give a map or fingerprint of molecular features and process conditions critical to maintain or reproduce the structure and function of the biologic. The resulting map can serve as a development tool, e.g., as a guide or target during the development of expression, purification, and formulation protocols, after development as a quality assurance tool, or as a definitive identifier of the specific biologic. It is contemplated that the map may serve as the definition of the biologic and provides the means by which a given product may be reliably reproduced and/or characterized as a biosimilar of another biologic product.

More specifically, during the first phase, various features of the structure, for example, the fine structure of the biologic are determined. The biologic is known (or expected, based for example on data generated through at least pre-clinical studies) to exhibit a desired biological activity thought to support some prophylactic or therapeutic utility, i.e., it comprises at least a lead compound. This phase typically operates on a reference biologic. By exploiting recently developed, known analysis techniques, the invention contemplates, as one step, characterizing the molecule, e.g., its primary, secondary, tertiary and/or quaternary structures. It determines such things as the precise post-translational peptide sequence, the pattern of inter and intra-chain disulfide bonding including the presence of any disulfide knots, whether and where the molecule is glycosylated, the pattern of the glycosylation, whether the molecule is truncated after expression, whether its activity is dependent on covalent or non-covalent dimerization, and generally, the nature of its post-translational modification(s). This phase typically employs mass spectroscopy-based protein analysis techniques, and typically may involve other analytical techniques such as electrophoresis, selective proteolysis, UV spectra analysis, Fluorescent spectra analysis, IR spectra analysis and MRI spectra analysis.

During the second phase, the reference biologic is subjected to various conditions that stress its molecule structure and may result in its modification, degradation, denaturation, oxidation, deamination, contamination, or aggregation, etc. Thus, for example, the molecule is subjected to different temperatures, different pH values, different ionic environments, enzymes that are commonly present in production broths, lyophilization and reconstitution conditions, mechanical stresses such as filtration and other separation techniques, and/or other conditions that the molecule might encounter during expression, purification, formulation, storage, or in vivo, such as physiological temperature, or various biomolecules (e.g., proteases), ions, or dissolved gases. The as-stressed molecule, and/or its fragments, are themselves analyzed to determine the effect, if any, of the stress on its fine structure. Thereafter, the structures are compared to that of the intact, unmodified molecule, and optionally, activity assays are done on as stressed molecular species which exhibit an alteration in structure.

This phase results in the identification of vulnerable substructures of the protein, i.e., identify and characterize parts of the molecular structure at risk of modification, degradation or aggregation when exposed to various physical and chemical conditions during the life of the biological drug. This is, in essence, a detailed stability study. Thereafter, it is determined which of these potentially labile features are material to efficacy, safety or potency (and inferentially, which are immaterial). Furthermore, the material vulnerable molecular features optionally are correlated to the physical and/or chemical conditions which threaten their modification.

In the third phase, the resulting information is compiled into a map of the specific molecular attributes that are computationally or statistically shown to impart the desired function to the molecule, and/or those attributes or areas that do not impart function, and optionally that specifies the conditions of expression, purification, formulation or storage necessary to maximize the chance of producing safe and efficacious drug meeting the structural aspects that are critical for its stability and biological activity. Thus, stability data is tied to efficacy data and also to processing data, resulting in a data set constituting a biological map that uniquely characterizes the biologic.

The resulting map reveals which particular attributes and/or modifications affect the molecule's activity, those attributes and/or modifications that do not significantly affect the molecule's activity (in other words, that are innocuous), and what specific conditions induce modifications, degradation or aggregation. The map thus enables determination of which processing parameters degrade or risk degrading the key structural features of the biologic known to be critical for its function and safety. Stated differently, the map enables determination of (a) which structural features of the protein are susceptible to alteration or degradation, for example caused by selection of particular raw materials used and/or processing steps employed in its expression or purification, and/or (b) which attributes or structural features of the molecule are necessary to impart the desired function of the molecule, and almost as importantly, which don't.

The following sections discuss the preparation of the reference biologic, the characterization of the reference biologic under native and under as-stressed conditions, the identification of structural features or attributes that are necessary to impart the desired function of the molecule, and that are important to the safety, purity, potency and/or efficacy of the biological molecule, the creation of a map identifying such structural features, and the use of such a map during the development, manufacture, purification, formulation and storage of such a biologic.

### (I) Structural Characterization of Reference Biologic

### (I) Sample Preparation

The structure of biological molecules can be very complex. With proteins or glycoproteins, for example, any modification to the backbone amino acids (for example, by deamidation, oxidation, methylation, acetylation, and isomerization etc.), changes in disulfide bonds (for example, shifting to cause mispaired cysteine bridges or unpaired cysteine residues), changes in glycosylation (for example, glycosylation site and glycan pattern) or the post translational modification can cause changes in protein folding or stability leading to loss of biological function.

### (a) Purification

It is contemplated that, for a biologic of interest, the molecule preferably has a purity of at least 50%, i.e., the molecule comprises at least 50% by weight of all of the components in a given sample. If the purity is less than 50%, the molecule preferably is purified to remove impurities (for example, process-related contaminants, unrelated biological macromolecules, misfolded proteins, etc.) using conventional purification techniques known in the art. For example, the molecule can be purified from other macromolecules present in a cell sample (for example, unrelated nucleic acids, lipids, glycolipids, polysaccharides, lipopolysaccharides, proteins, or even misfolded and/or misprocessed forms of the biological molecule of interest) (Conlon et al., METHODS MOL BIOL. (2012) 917: 369-390). Conventional purification techniques can be used, which include immunocapture (also known as immunoaffinity) and non-antibody based purification approaches Immunoaffinity uses an antibody or a ligand that selectively binds the biological molecule of interest (Urh et al., METHODS IN ENZYMOLOGY (2009) Volume 463, Chapter 26, pp. 416-438; Moser et al., BIOANAYLSIS (2010) 2(4): 769-490). This technique can be very effective in selecting the molecule of interest from a complex mixture, for example, an extract.

Non-antibody-based protein purification protocols can include, but are not limited to, protein precipitation (Brugess, METHODS IN ENZYMOLOGY (2009) Vol. 463, Chapter 20, pp. 331-342), gel filtration (Stellwagen, METHODS IN ENZYMOLOGY (1990) Vol. 463, Chapter 23, pp. 373-385), ion exchange chromatography (Jungbauer et al., METHODS IN ENZYMOLOGY (2009) Vol. 463, Chapter 22, pp. 349-371), and gel electrophoresis (Garfin, METHODS IN ENZYMOLOGY (1990) Vol. 463, Chapter 29, pp. 497-513; Friedman et al., METHODS IN ENZYMOLOGY (2009) Vol. 463, Chapter 30, pp. 515-540). Protein precipitation methods utilize solvents (for example, ammonium sulfate, polyethyleneimine, acetone, acetonitrile, or ethanol, etc.) to remove unwanted small molecules by precipitating the molecules of interest and/or by precipitating unwanted molecules. Size-based fractionation can be achieved by gel filtration (for example, by size-exclusion chromatography) where molecules are separated based on differences in molecular size during passage through a porous medium packed in a gel filtration column. Ion exchange chromatography utilizes charge differences between a stationary phase and a mobile phase to purify the molecule of interest. For example, positively charged proteins can be isolated using a cation exchange chromatography where the stationary phase is negatively charged. Negatively charged proteins can be separated using an anion exchange chromatography in which stationary phase is positively charged. Gel electrophoresis, for example, one-dimensional gel electrophoresis (Garfin (1990) supra), or two-dimensional gel electrophoresis (Friedman (2009) supra) fractionates molecules based on size, shape, and charge. The molecule of interest, such as, a monoclonal antibody, can be isolated by sodium dodecyl sulfate (SDS) polyacrylamide gel under non-reducing and/or reducing conditions. It is understood that a number of purification techniques may be used in combination to achieve the requisite purity.

After fractionation, the molecules can be visualized by standard staining approaches (for example, using SimplyBlue™ SafeStain or SilverQuest™ Silver Staining Kit, both available from Life Technologies). The unbound stain can be removed by rinsing the gel with deionized water and/or ammonium bicarbonate buffer until the gel has no stain (for example, blue stain) background color. The gel bands of interest, once identified, are cut from the gel and dried with acetonitrile using a CentriVap vacuum concentrator. The molecules in the gel bands can be harvested from the gel band or treated or analyzed further within the gel, for example, exposed to in-gel digestion.

### (b) Denaturation

During the characterization process, the molecules of interest may be denatured under a variety of conditions where the molecules unfold and lose their secondary, tertiary, and/or quaternary structures. The primary structure of protein is based upon the amino acid sequence of the protein. Functional proteins typically are fold into a high order structure involving secondary structures (for example, via hydrogen bonding between peptides to form α-helices and β-sheets), tertiary structures (for example, through hydrophobic interactions, ionic interactions, and disulfide bridges to produce a three-dimensional protein molecule), and quaternary structures (for example, by non-covalent interactions and disulfide bonds to connect subunit proteins) (Jones, ADVANCED DRUG DELIVERY REVIEWS (1993) 10:29-90).

In order to characterize higher order structures, the molecule of interest can be subjected to denaturation, which disrupts secondary, tertiary, and quaternary structures. Denaturation can involve exposure to elevated temperatures (Freire, METHODS IN ENZYLMOLOGY (1995) 259: 144-168; Friedman (2009) supra), exposure to chemical denaturants (Neurath et al., CHEM. REV. (1944) 34(2): 157-265; Makhatadze et al., J. MOL. BIOL. (1992) 226: 491-505), and exposure to mechanical stress, for example, freeze-thaw processes (Pikal-Cleland et al., ARCHIVES OF BIOCHEMISTRY AND BIOPHYSICS (2000) 384(2): 398-406).

Thermal denaturation (for example, heat at 100° C. for 10 minutes), can disrupt hydrogen bonds occurring between amide groups in the secondary protein structure and hydrophobic interactions in the tertiary protein structure. Chemical denaturation may include the use of organic solvents (for example, cleavage of hydrogen bonds in secondary and tertiary structures by acetonitrile, methanol, or ethanol), acids (for example, dissociation of ionic interactions by formic acid, trichloroacetic acid, hydrochloride), and chaotropic agents (for example, disruption of hydrophobic interactions in tertiary and quaternary structures by urea, guanidine hydrochloride, sodium dodecyl sulfate). Mechanical stress (for example, freezing-thawing process) can also induce protein denaturation in solution or lyse a cell sample by disrupting the cell through ice crystal formation.

### (c) Reduction and Alkylation

The molecule of interest may contain a disulfide bond (S—S) (also known as a disulfide bridge), that is formed by coupling two thio groups (—SH) from cysteine residues (Mullan et al., BMC PROCEEDINGS (2011) 5 (Suppl 8): 110). The disulfide bridges can include intra- and/or inter-chain disulfide bonds. For example, monoclonal antibodies comprise two light (L) chains and two heavy (H) chains that are covalently linked by inter-chain disulfide bonds. In addition, intra-chain disulfide bonds can be found in variable (V) and constant (C) domains on each light and heavy chain. Mis-paired disulfide bonds including unpaired cysteines (for example, occurring to pH change during manufacturing purification process or storage) can result in loss of biological activity such as drug efficacy, for example, as typically occurs with monoclonal antibodies. Therefore, it is important to determine the location of intra-chain and inter-chain disulfide bonds and their status (for example, unpaired, mispaired, or scrambling).

Disulfide bridges can be cleaved using reducing agents such as 2-mercaptoethanol (Jocelyn, METHODS IN ENZYMOLOGY (1987) 143:246-255), dithiothreitol (DTT) (Jocelyn (1987) supra), and tris(2-carboxyethyl)phosphine (TCEP) (Getz et al., ANALYTICAL BIOCHEMISTRY (1999) 273: 73-80). For example, during a reduction reaction, the molecule of interest can be subjected to a solution containing 50 mM DTT at 37° C. for 30 minutes to break cysteine bridges by reducing the disulfide bonds. To prevent reformation of disulfide bonds after protein reduction, opened cysteine residues (or called free thiol groups, —SH) are capped using an alkylating agent such as iodoacetic acid, iodoacetamide (IAA), or N-ethylmaleimide (NEM) (Anfinsen et al., J. BIOL. CHEM. (1961) 236: 1361-1363). For example, 100 mM IAA is commonly added to protein solution after completion of DTT reduction and then kept in dark at room temperature for 45 minutes.

### (d) Fragmentation

Depending upon the characterization methods to be used, the molecule of interest can be fragmented using a variety of techniques, which can be executed in solution (solution digestion), in a gel (in-gel digestion), or in a gas phase (gas-phase fragmentation). In general, enzymes and chemicals can be utilized to cleave molecules, for example proteins, in solution and in gel. Electric forces can be induced in the gaseous phase of an electronic instrument (for example, a mass spectrometer) to fragment molecules of interest.

### (i) Enzymatic Digestion in Solution

Exemplary proteolytic enzymes include trypsin, Lys-C, Glu-C, Asp-N, and Arg-C, in which cleavage sites are highly specific. Exemplary deglycosylating enzymes release glycans from glycoproteins, which can include N-glycosidases such as peptide N-glycosidase F (PNGase F), O-glycosidase, sialidase, glucosaminidase, and β-galactosidase, etc. (Jensen et al., NATURE PROTOCOLS (2012) 7(7): 1299-1310). These enzymes are commonly used to generate various sizes of peptides and/or oligosaccharides. For examples, trypsin cleaves the carboxyl side of lysine (Lys) and arginine (Arg) residues if the next residue is not proline (Pro). Lys-C hydrolyzes the peptide bond at the carboxyl side of Lys. Glu-C cuts the carboxyl side of glutamate (Glu). Arg-C cleaves the C-terminal side of Arg residues including the site next to Pro. Asp-N breaks peptide bonds on the N-terminal side of aspartic acid (Asp) residues. PNGase F hydrolyzes the amide bond of asparagine (Asn) to Asp residues and releases oligosaccharides from N-linked glycoproteins. Less specific enzymes such as pepsin, papain, chymotrypsin, aminopeptidases, carboxypeptidases can also be used to produce fragments depending of the structural complexity of interest. (Switzar et al., J. PROTEOME RES. (2013) 12: 1067-1077). For example, pepsin cleaves monoclonal antibodies (mAb) into F(ab)₂ fragment and papain typically breaks mAb into two Fab fragments and an intact Fc fragment.

To obtain a full coverage of a molecule structure of interest, it can be necessary to use several enzymes, which can be introduced as single enzymes in a series of separate digestions, or can be used as a mixture of enzymes.

For multiple single enzymes in a serial process, for example, trypsin, is added to a protein solution (1:50 w:w) in 100 mM ammonium bicarbonate or 100 mM tris-HCl buffer (pH 6.5 to 8), and incubated at room temperature or at 37° C. After 4-hour incubation, trypsin can be added to a protein solution (1:50 w:w) again and incubated for another 16 hours. Formic acid, 5%, can be used to stop the enzymatic reaction. Then, the trypsin digest is subjected to exchange buffer with 10 mM HCl (pH 2) before pepsin digestion (pepsin:protein 1:10 w:w) is conducted at 37° C. for 30 minutes. The pepsin reaction can be terminated by adjusting pH to 5 with ammonium bicarbonate buffer. The resulting digest then is subjected to a buffer exchange with 100 mM ammonium bicarbonate (pH 8) if the digestion solution is subjected to another digestion. For example, PNGase F (1 unit/10 μg) is added to pepsin digestion solution after buffer exchange. Deglycosylation reaction is carried out at 37° C., up to 24 hours. Formic acid, 5%, can then be added to stop the enzymatic reaction.

Alternatively the molecule of interest can be exposed to multiple enzymes in a single reaction mixture if digestion conditions such as optimal pH ranges for each individual enzyme are similar, for example, a cocktail mixture of (trypsin and Lys-C), (trypsin, Lys-C, Asp-N), or (trypsin, Lys-C, Asp-N, PNGase). If pepsin is added for multiple enzyme digestion, a serial process is required. This is because pepsin is only active under an acidic condition (pH 2). For example, if a solution containing a monoclonal antibody is subjected to pepsin digestion in 10 mM HCl (pH 2, 37° C., 30 minutes), buffer should be exchanged, for example, with 100 mM ammonium bicarbonate at room temperature using 10,000 molecular weight cut-off membrane filters, before continuing the digestion with trypsin (1:50 w:w) and/or Lys-C (1:50 w:w).

### (ii) Chemical Digestion in Solution

Chemicals useful in fragmenting proteins include cyanogen bromide (CNBr) (Zhang et al., ANAL. CHEM (1996) 68(19): 3422-3430), 2-nitro-5-thiocyanobenzoate (NTCB) (Tang et al., ANAL. CHEM. (2004) 334: 48-61), hydroxylamine (Bornstein et al., METHODS ENZYMOL. (1977) 47: 132-45), and formic acid (FA) (Landon, METHODS IN ENZYMOLOGY (1977) 47: 145-149) etc. CNBr breaks peptide bonds at the C-terminal side of methionine (Met) residues. NTCB cleaves proteins at cysteine (Cys) residues through reactions of cyanylation and β-elimination. Hydroxylamine hydrolyzes asparaginyl-glycyl (Asn-Gly) peptide bonds. Formic acid cuts proteins at aspartic acid-proline (Asp-Pro) peptide bonds.

Similar to enzymatic digestion, multiple chemicals can be included in the same reaction mixture provided that the reaction conditions are compatible with each chemical. For example, CNBr reaction is often coupled with 70% formic acid or 70% trifluoroacetic acid (TFA) in dark at room temperature for 16 hours (Zhang (1996) supra).

To increase coverage for structural characterization, a combination of enzymes and chemicals can be subjected to protein of interest (Kwon, A JOURNAL OF CHROMATOGRAPHY (2010) 1217: 285-293). For examples, proteins can be submitted to acid hydrolysis using 25% formic acid and incubated at 95° C. for 4 hours. After buffer exchange with 100 mM ammonium bicarbonate (pH8), the protein solution can be further treated by a single enzyme or multiple enzymes.

Fragments obtained from solution digestion can be directly submitted for structural analysis by mass spectrometry. However, extraction (for example, solid phase extraction or liquid/liquid extraction) may be required to improve the recovery of peptides generated from solution digestion.

(iii) In-Gel Digestion with Enzymes and/or Chemicals

The molecules of interest, when captured in a gel band, can be subjected to digestion in situ. For example, the gel bands of interest are cut into approximately 1×1 mm slices, and then are subjected to digestion (in-gel digestion). Similar to solution digestion, enzymes and/or chemicals can be used to cleave proteins of interest in each gel slice. In-gel digestion can be conducted with or without in-gel reduction and alkylation (Shevchenko, NATURE PROTOCOLS (2006) 1(6): 2856-2860). For example, if a gel band is excised under a non-reducing condition, dried gel pieces are covered with 10 mM DTT in ammonium bicarbonate buffer and incubated at 56° C. for 30 minutes. Then 55 mM IAA is added to cover the gel pieces in dark at room temperature for 45 minutes after removing the remaining DTT solution. The reduced and alkylated gel samples then are dried with acetonitrile using a CentriVap vacuum concentrator, followed by washing the samples with ammonium bicarbonate buffer and deionized water Ammonium bicarbonate buffer containing enzymes and or chemicals is added to cover the dried gel bands. The remaining buffer containing enzymes and/or chemical is removed after incubating at 4° C., up to 1 hour. Blank buffer without enzymes or chemicals then is added to cover the gel samples and incubate at 37° C. for overnight.

Fragments obtained from in-gel digestion are typically extracted with 5% formic acid and acetonitrile (1:2 v:v) and then dried down using a CentriVap vacuum concentrator to yield approximately 10 μL of wet extract for each gel digest prior to mass spectrometric analysis.

### (2) Characterization of the Reference Biologic

Mass spectrometry is the most suitable and useful tool to be used for characterization of complex biologics (Mann, ANNU. REV. BIOCHEM. (2001) 70: 437-473; Jardine, METHODS IN ENZYMOLOGY (1990) 193: 441-455). This is because mass spectrometers directly read the mass fingerprints (mass/charge ratios, m/z) of intact or fragmented proteins or molecules. Four types of mass spectrometers, quadrupole, ion trap, time-of-flight (TOF), and fourier transform ion cyclotron resonance (FTICR), have been widely used to obtain structural information for biomolecules. Modern hybrid mass analyzers such as a hybrid linear ion trap-obitrap (e.g., Thermo LTQ Obitrap Elite) and a quadrupole-time-of-flight (e.g., Agilent Q-TOF) have been developed for structural characterization of biologics to support biopharmaceutical discovery and development pipelines. Fragmentation techniques (gas-phase fragmentation) used on mass analyzers include collision induced dissociation (CID), higher-energy collisional dissociation (HCD), electron capture dissociation (ECD), electron transfer dissociation (ETD), infrared multi-photon dissociation (IRMPD), and CID of the isolated charge-reduced ions followed by ETD (CRCID), depending on the type of mass spectrometers used (Scigelova, PRACTICAL PROTEOMICS (2006) 1-2: 16-21; Elviri, TANDEM MASS SPECTROMETRY—APPLICATIONS AND PRINCIPLES (2012) 162-178). For example, CID, HCD, and ETD are built on a hybrid linear ion trap-obitrap mass spectrometer; CID and ECD are constructed on FTICR MS; low-energy CID is configured on Q-TOF MS; high-energy CID is included on TOF/TOF MS.

Typically, CID can be used for small (for example, peptides 15-20 amino acid residues in length), low charge (for example, +1, +2, +3 charged state) and unmodified peptides. A low-energy CID fragmentation occurs at amide bonds of the peptide bone to generate typically characteristic b and y sequencing ions, which are particularly suitable for peptide sequencing. CID fragmentation depends on the protein or peptide sequence, the peptide length, or the presence of post-translational modifications (PTMs). For example, a peptide having several basic amino acid residues can prevent random protonation on the peptide backbone inducing site specific dissociation and few sequence ions. Certain post-translational modifications can prevent the random protonation on the peptide backbone and subsequently inhibit CID fragmentation.

ECD is based on the gas phase fragmentation of multiple charged protein and peptide ions upon capture of a low energy electron within a mass analyzer such as FTICR MS. The ECD fragmentation can take place through the cleavage of N-Cα bond on the peptide bone to generate c and z ions series of peptide fragments (Elviri (2012) supra) and is able to retain post-translational modifications. However, a large amount of pure sample is required for this approach.

ETD fragmentation involves in a proton-electron transfer process between large peptides or proteins and reagents (for example, transfer an electron from a radical anion to a protonated peptide) (Elviri (2012) supra). Through a proton-electron transfer, peptide backbone N-Cα bonds can be fragmented into c and z sequencing ions without dissociating side chain of amino acid residues. ETD does not require a large amount of sample to be used for MS analysis. Both ECD and ETD are independent of the peptide length and amino acid compositions, which are suitable to fragment an intact protein, large peptides, or labile proteins with post-translational modifications.

To permit the introduction of biologics, such as proteins and peptides, into a mass spectrometer, an electrospray ionization (ESI) or matrix-assisted laser desorption ionization (MALDI) sources are the most common interfaces employed on mass spectrometers. The ESI interface enables on-line introduction of samples (analytes) using HPLC, CE, or infusion pump to deliver analytes from solution phase into gas phase on a mass analyzer. The MALDI interface is especially beneficial for a sample where the amount is limited. For example, a protein or peptide sample (1 μL) typically is spotted on a MALDI plate having a matrix such as α-cyano-4-hydroxycinnamic acid or sinapinic acid to form a crystal prior to MS analysis. Mass spectrometric methods using ESI ionization with CID, HCD, ETD, and CRCID fragmentation techniques can be used to facilitate structural characterization of biologics.

### (a) Molecular Weight

Molecular weight is an important aspect of biologics of interest. Conventional techniques used to measure molecular weights of proteins include, but not limited to, gel filtration chromatography (“Gel Filtration. Principles and Methods” (2002) supra; Laue et al., METHODS IN ENZYMOLOGY (1990) 182:566-587), (or called size exclusion chromatography, SEC), electrophoresis (Jardine (1990) supra; Laue (1990) supra), light scattering (Harding, METHODS IN MOLECULAR BIOLOGY (1994) 22:85-95), analytical ultracentrifugation (Harding, METHODS IN MOLECULAR BIOLOGY (1994) 22:75-84) and mass spectrometry (Jardine (1990) supra; Siliveira (2009) supra; Wysocki (2004) supra; Scigelova (2006) supra; Elviri (2012) supra; Laue (1990) supra), (MS) etc. SEC and gel electrophoresis provide relative molecular weights based upon comparison with molecular weight standards. Absolute molecular weights can be measured by light scattering, analytical ultracentrifugation, and mass spectrometry.

For example, an intact protein (1 mg/mL) in solution after purification can be analyzed directly by a mass spectrometer (for example, Thermo Q Exactive Plus) equipped with an ESI interface to determine molecular weight. The molecular weight of the protein can be obtained after deconvolution of multiple charged states of intact protein using Thermo “Protein Deconvolution” or “PepFinder” software. A combined technique, for example, SDS PAGE-MS (Schuhmacher et al., ELECTROPHORESIS (1996) 17:848-854), capillary electrophoresis-MS (Haselberg et al., ELECTROPHORESIS (2011) 32:66-82), or high pressure liquid chromatography-MS (Shi et al., JOURNAL OF CHROMATOGRAPHY (2004) 1053: 27-36), is utilized to determine MWs of biologics if the conditions of biological references are complicated (for example, purity<50%) or not in favor for MS analysis (for example, containing high salts). For example, SDS PAGE can be carried out to purify the molecule of interest from a mixture (e.g., impurities), and then the purified molecule is subjected to MW determination by a mass analyzer. Heterogeneous glycoforms of mAb, separated by CE or HPLC are identified through their MWs by MS. SEC-light scattering, can be employed to measure the sizes and the molecular weights of proteins, including protein aggregates (Arakawa et al., BIOPROCESS INTERNATIONAL (2006) 4(10): 42-43; Arakawa et al., BIOPROCESS INTERNATIONAL (2007) 36-47).

### (b) Protein Primary Structure

The primary structure of protein refers to the amino acid sequence of the polypeptide. It is very important to confirm the amino acid sequence of the protein backbone since amino acid modifications may occur during manufacture or storage of biologics, which can result in the loss of stability and/or biological function. A peptide map of amino acid sequence on proteins including any amino acid modifications is one of major quality characteristics for protein biologics. Sequencing can be accomplished using a variety of approaches.

Edman degradation is a traditional method used in peptide sequencing (Schroeder, METHODS ENZYMOL. (1967) 11:445-461; Niall, METHODS ENZYMOL. (1973) 27:942-1010). Chemical reagents such as phenylisothiocyanate (PITC) can couple with the N-terminal amino group of a protein or peptide to form a phenylthiocarbamyl (PTC) adduct which can be cleaved under acidic conditions. Due to the limitation of Edman degradation reactions, which have to proceed from the N-terminal of peptides, this method cannot be used to sequence peptides on protein where N-terminal amino acid is modified. Thus, mass spectrometry is the preferred method for peptide sequencing.

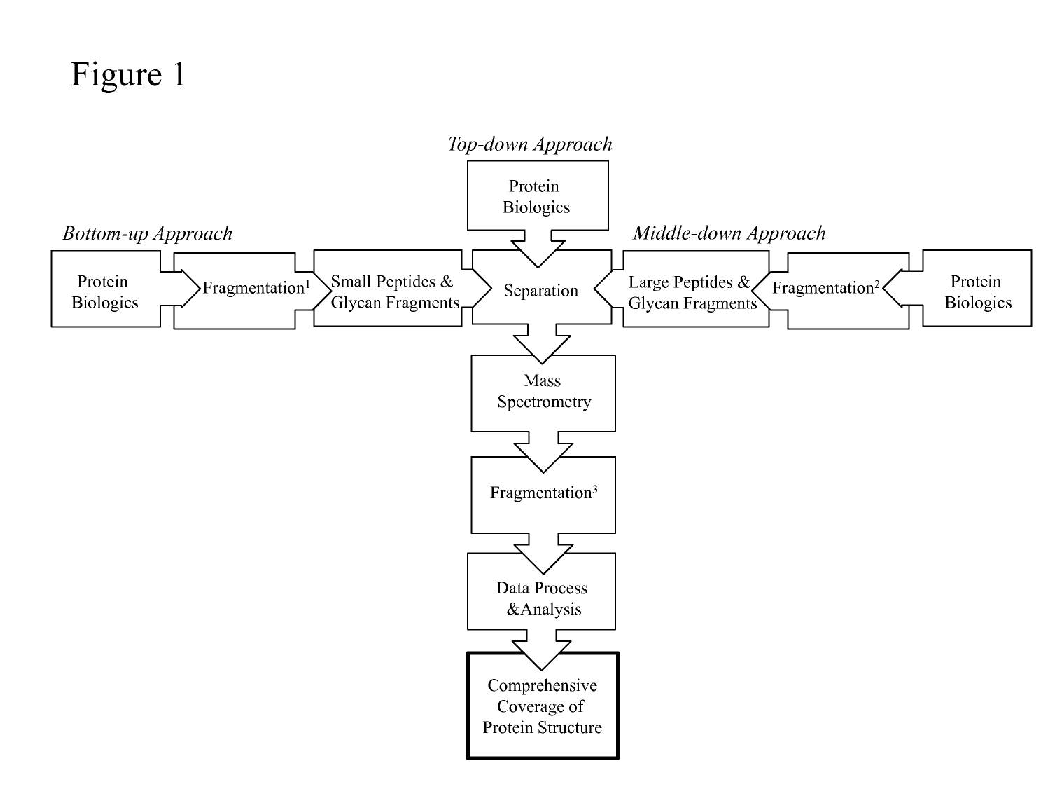

Mass spectrometry-based platforms (e.g., bottom-up, middle-down, and top-down) using multiple fragmentation techniques can be used to obtain a comprehensive coverage of peptide sequence on protein biologics (see FIG. 1). In the bottom-up approach, which serves as a standard method for peptide sequencing, a protein is digested into small peptide fragments (Wysocki (2004) supra; Scigelova (2006) supra; Wu et al., JOURNAL OF PROTEOME RESEARCH (2007) 6: 4230-4244). These small peptide fragments then are identified by MS/MS typically using CID. Nevertheless, the whole protein sequence can still be uncertain, mainly because of redundant peptide sequences present in the protein or loss of labile post-translational modifications. The middle-down approach is an intermediate method to the bottom-up approach (Wu et al. NAT. METHODS (2013) 9(8): 822-824). A protein is digested into large peptide fragments, which are subsequently fragmented using CID and/or ETD, on a mass spectrometer. In general, use of bottom-up and middle-down approaches covers most of peptide sequences on proteins. Yet, a full coverage of protein sequence may not be possible for some proteins. A top-down approach can be used as an alternative to bottom-up and middle-down methods (Wysocki (2004) supra; Scigelova (2006) supra). An intact protein without digestion can be directly measured by mass analyzers and subsequently fragmented by CID, HCD, and ETD. Top-down sequencing allows locating post-translational modifications and differentiating isomers which could be lost in bottom-up and/or middle-down approaches. The use of two or more of these approaches of mapping peptide fragments enables high probabilities of a full coverage of peptide sequences on proteins.

For bottom-up sequencing, a protein is subjected to digestion using a single enzyme such as trypsin and multiple enzymes such as (trypsin and Lys-C), (trypsin and Asp-N), or (trypsin, Lys-C, and Asp-N), etc. to generate many small peptide fragments in solution (see the section of sample preparation for details). Then the digested protein containing a mixture of small peptide fragments is subjected to HPLC separation and subsequent MS/MS analysis.

Two types of HPLC separation can be performed depending on the sensitivity of peptides. If sensitivity is not a concern, the digested protein (for example, 10 μg) is subjected to a reverse-phase LC separation using a C18 column (for example, Agilent 300SB C18 column, 2.1 mm id×15 cm). Normally, mobile phase A contains 0.1% formic acid in water and mobile phase B consists of 0.1% formic acid in acetonitrile. The separation of peptides is achieved through a gradient (for example, from 2% mobile phase B to 40% B in 60 minutes at a flow rate of 200 μL/min) using a HPLC pump (for example, Agilent 1200 HPLC system or Dionex UltiMate 3000 RS pump) coupled with a mass spectrometer. Followed by HPLC separation, peptides are introduced into a mass analyzer through an electrospray interface under a positive or a negative ionization mode. If sensitivity becomes an issue due to the limited protein source or a poor recovery of peptides digested from a protein, HPLC separation can be carried out using a nano-capillary C18 column (for example, Michrom Bioresources Magic C18 or Thermo Acclaim PepMap RSLC C18 column, 100 Å pore, 2 μm, 75 μm id×15 cm) on a nano-capillary HPLC pump (for example, Dionex UltiMate 3000 RSLCnano pump). The separation of peptides on a nano-capillary column is accomplished using a gradient (for example, from 2% to 60% B in 90 minutes) at a flow rate of 200 nL/min. A nano-spray ion source (for example, Thermo Nanospray Flex) is equipped with a mass spectrometer (LTQ obitrap Elite ETD) to introduce the sample after nano-capillary HPCL separation.

Once peptides are delivered into a mass spectrometer, peptides are positively charged (known as precursor ions) and subsequently fragmented by applying CID energy to produce smaller fragments (known as product ions). For example, LTQ Obitrap Elite ETD can be operated under a data-dependent mode to allow automatically switch between MS, CID-MS², ETD-MS², and/or HCD-MS². Mostly, MS, CID-MS², and ETD-MS² are sufficient for peptide sequencing. CID on an isolated charge-reduced species (i.e., CRCID) generated from ETD-MS² is normally used to characterize phosphorylation, disulfide bond formation, and glycosylation. After a survey full-scan MS spectrum (for example, from m/z 400 to 2000), subsequent CID-MS² and ETD-MS² activation scan steps can be performed on the same precursor ion (for example, a peptide fragment generated in solution) over the same m/z scan range as that used for the full-scan MS spectrum. The precursor ion is isolated using the data-dependent acquisition mode with a ±2.5 m/z isolation width to select automatically and sequentially a specific ion from the survey scan. Then, an additional CID-MS³ step is performed on an isolated precursor ion with a ±5 m/z isolation width and with the highest intensity from the CID-MS², ETD-MS², or HCD-MS² scan. CID-MS², ETD-MS², and HCD-MS² can be repeated in sequence to select for fragmentation of subsequent highest intensity precursor ions from the first survey scan. The peptide sequence can be identified by assembling various types of fragment ions, for example, b and y ions mainly produced by CID-MS²; c and z ions generated by ETD-MS² for each peptide with assistance of software (see assignment of peptide sequence for details) (Steen et al., MOLECULAR CELL BIOLOGY (2004) 5:699-711).

In the middle-down approach, the protein is subjected to digestion using a protease (for example, Lys-C, Asp-N, or Glu-C) or a chemical (for example, CNBr) to generate large peptide fragments in solution. Then the digested protein containing a mixture of large peptide fragments is subjected to HPLC separation and subsequent MS/MS analysis.

In the top-down approach, the intact protein is subjected to nano-capillary LC MS/MS analysis using multiple fragmentation techniques (for example, CID, HCD, and ETD). The fragmentation spectra generated from an intact protein are more complicated, compared to the bottom-up and the middle-down approaches. To avoid collecting muddled data from a mixture of proteins, the protein of interest usually needs to be purified or fractionated (for example, GELFREE™ fractionation; Tran et al., ANALYTICAL CHEMISTRY (2008) 80(5): 1568-1573) prior to MS analysis. After purification or fractionation, the protein sample is separated using a nano-capillary HPLC column (e.g., 1000 Å, 5 μm, polymer reversed phase PLRP-S, 75 μm id×10 cm). CID, HCD, and ETD fragmentation are applied to the most intense charge state of an intact protein on the Thermo LTQ Obitrap Elite ETD mass spectrometer.

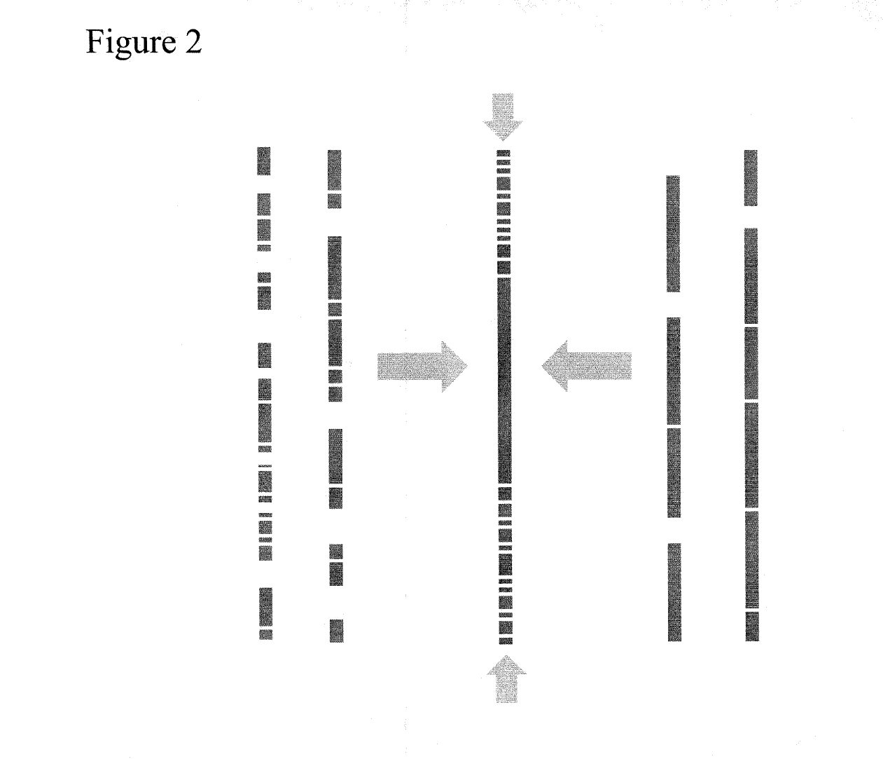

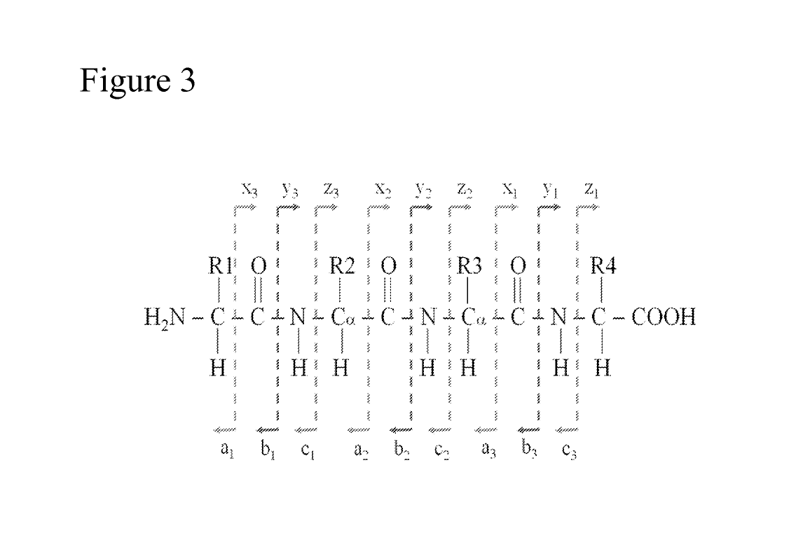

The protein or peptide sequence can be created using a variety of approaches that assign the peptide sequences. A staggered strategy of mapping fragments obtained from bottom-up, middle-down, and/or top-down methods, as shown in FIG. 2, is used to confirm the protein sequence. Based on the fragmentation pattern (for example, a, b, c, x, y, z ions, in FIG. 3) observed from MS/MS spectra, the sequence of each peptide fragment can be assigned using software(s) to obtain theoretical digested peptide masses to match with the experimental data (i.e., MS/MS fragmentation spectra). For example, MS/MS spectra (bottom-up and middle-down sequencing), generated on a LTQ Obitrap Elite ETD and a Q Exactive Plus mass spectrometers are processed using Thermo PepFinder software that has the predicted MS/MS algorithm incorporated to assign fragmentation spectra to the most probable peptide sequence. The spectra generated in the CID-MS², ETD-MS², HCD-MS² and/or CID-MS³ are searched against spectra of theoretical fragmentations (for example, b and y ions) using PepFinder to assign peptide identification based on the mass accuracy, the similarity of MS/MS pattern, status of disulfide bond (reduced or non-reduced), N-glycosylation (CHO N-glycan, human N-glycan, or none), mass changes for unspecified modifications, and statistical confidence (for example, 90%) etc. Final confirmation of the most probable peptide sequence assignments is obtained by inspection of individual mass spectra with the preferred fragmentation patterns in the observed CID-MS², ETD-MS², HCD-MS², and/or CID-MS³ spectra. The peptide assignment for top-down MS/MS spectra can be performed using Mascot (Matrix Science) or ProSight Suite (Thermo Fisher Scientific).

### (c) Amino Acid Modifications

Amino acid modifications can occur to biological molecules during manufacture, formulation, or storage as a consequence of protein degradation or post-translational modifications. Protein degradation can occur as a result of chemical and physical modification. Chemical modification can change peptide backbone amino acids through oxidation, deamidation, isomerization, and racemization. Physical modification can trigger unfolding, misfolding or aggregation on proteins. Post-translational modifications usually happen during production of biologics in a cell or a cell system. Common post-translational modifications include acetylation, methylation, phosphorylation, and glycosylation, which may take place in N-terminal, C-terminal, or side chain of amino acid residues. The altered or modified amino acids can be detected via peptide sequencing using enzymes, chemicals, and MS fragmentation techniques, as described above. The key for identifying amino acid modifications is to locate modification sites which can be found according to mass differences between modified (observed MS spectra) and un-modified (theoretically predicted MS spectra) amino acids. To confirm the modification sites, the peptide containing modified amino acid(s) is subjected to MS/MS analysis. Use of MS/MS methods depends upon the modification site of interest. Examples of characterization of amino acid modifications are illustrated below.

### (i) N-Terminal Modifications

N-terminal modifications include, but not limited to, acetylation, methylation, formylation, cyclization of glutamine, myristoylation, phosphorylation, and glycosylation, etc. (Meinnel et al., PROTEOMICS (2008) 8: 626-649). For example, acetylation takes place mostly at a lysine (Lys) residue; formylation is often observed on an N-methionine (Met) residue; cyclization converts glutamine (Gln) to pyroglutamic acid (pGlu) which is observed in mAb; myristoylation usually occurs to a glycine (Gly) residue. Based on the type of enzyme chosen for digestion, the digested protein containing peptide fragments including N-terminal peptide is subjected to LC-MS/MS analysis. The mass of the N-terminal peptide with a modification on amino acid can be monitored by LC-MS on a mass spectrometer. The type of N-terminal modification can be distinguished by mass differences (for example, +42 for acetylation; +14 for methylation; +28 for formylation; +17 for cyclization of glutamine to pyroglutamic acid; +210 for myristoylation; +80 for phosphorylation) through the MS full scan followed by LC separation. Once detecting the predicted mass of the modified N-peptide, the N-peptide containing amino acid modification is selected as a precursor ion and then subjected to CID-MS² and/or ETD-MS² to produce more fragments. Generally, the modification on the N-amino acid residue can be detected by additional masses on b fragmentation ions observed from CID-MS² (for example, +42 for acetylation) and no mass shift on y fragmentation ions.

### (ii) C-Terminal Modifications

C-terminal heterogeneities often occur in recombinant monoclonal antibodies (Liu et al., JOURNAL OF PHARMACEUTICAL SCIENCES (2008) 97(7): 2426-2447). One of the most common C-terminal heterogeneities (Liu (2008) supra) is the incomplete C-terminal lysine processing of the heavy chain during production of monoclonal antibodies to produce three antibody species containing zero, one, and two C-lysine residues. To characterize C-terminal lysine modification, the antibody is subjected to digestion using enzymes and then separation of heavy chains from light chains using molecular weight cut-off centrifugation filters (for example, 10,000 Da cut-off). The heavy-chain fragments containing C-terminal processing lysine peptide species are subjected to LC separation (e.g., use of reverse-phase LC column), followed by MS/MS analysis. Generally, peptides containing heterogeneous C-terminal lysine residues can be separated by LC and then identified by MS. For example, a reduction of 128 Da in mass indicates a removal of one C-terminal lysine residue. The positive charge state on the removal of one C-terminal lysine peptide is decreased by 1 unit as well. Therefore, various C-terminal lysine species can be identified by LC-MS based on the charged state and the masses. C-terminal amidation species are also often noted in the heavy chain of monoclonal antibodies (Tsubaki et al., INTERNATIONAL JOURNAL OF BIOLOGICAL MACROMOLECULES (2013) 52: 139-147). Like C-terminal lysine modification, amidation (a reduction of 1 Da) occurs as a result of post-translational modification. Peptidylglycine α-amidating monooxygenase (PAM) can cleave C-terminal glycine (Gly) and amidate the penultimate amino acid, resulting in a reduction of 58 Da in mass if the second last amino acid to Gly residue is proline (Pro) or leucine (Leu). Similar to C-terminal lysine species, the heavy-chain peptide fragments are analyzed by MS using CID-MS² followed by LC separation, C-terminal amidation species can be identified.

(iii) Oxidation

Biologics can be oxidized if oxygen radicals or metals are present in the environment. In proteins, the most common oxidation occurs to amino acids containing a sulfur atom such as methionine (Met) and cysteine (Cys) or an aromatic ring such as histidine (His), tyrosine (Tyr), tryptophan (Trp), and phenylalanine (Phe) (Patal et al., BIOPROCESS INTERNATIONAL (2011) 20-31). For example, the sulfur atom (S) on Met reacts with oxygen radicals in solution to form methionine sulfoxide (S═O) and methionine sulfone (O═S═O). Cys oxidation opens disulfide links and forms new disulfide bonds, leading to mispaired disulfide bonds and scrambled disulfide bridges. Spontaneous oxidation (also known as auto-oxidation) may cause Cys to form sulfinic acid (SOOH) and cysteic acid (SOOOH) if metal ions are present in solution. His oxidation occurs through the reaction between imidazole rings to generate oxidation products such as 2-oxo-histidine (2-O-His), aspartic acid (Asp), and asparagine (Asn). Trp can be oxidized by light (also known as photo-oxidation) to form oxidation products such as N-formylkynurenine and kynurenine (Li et al., BIOTECHNOLOGY AND BIOENGINEERING (1995) 48: 490-500). Photo-oxidation of Tyr can form 3,4-dihydroxyphenylalanine (DOPA) and dityrosine, resulting in covalent aggregation through forming Tyr-Tyr cross links. Protein oxidation can be measured through LC-MS analysis of a protein digest. Use of theoretically predicted masses of peptides containing potential oxidation products (for example, +16 Da or +32 Da) and fragmentation pattern observed on peptide fragments can identify oxidation products on protein.

### (iv) Deamidation, Isomerization, and Racemization

Deamidation occurs to many recombinant proteins by removing an amide group from an amino acid such as asparagine (Asn) and glutamine (Gln) (Patal (2011) supra). Deamidation is a non-enzymatic process that can take place spontaneously on proteins or peptides in vivo or in vitro systems. Consequently, proteins undergo isomerization and racemization after deamidation. For example, Asn is initially converted to aspartic acid (Asp) by the non-enzymatic deamidation process, which can be identified through a mass shift of +1 Da on a mass spectrometer. Isoaspartic acid (isoAsp), as the most commonly found deamidation product, is then formed via isomerization of Asp. The isoAsp and Asp peptide products are normally separated by LC and subsequently identified by MS/MS. Besides, succinimide intermediate generated during Asn deamidation process can be converted to D-Asp (refer to racemization). Overall, the rate of deamidation on an intact protein is very slow; whereas the deamidation rate can be increased significantly for peptides under alkaline condition (Hao et al., (2011) MOLECULAR & CELLULAR PROTEOMICS 10.10). For example, D,L-Asp and D,L-isoAsp peptides (predominated with isoAsp peptides) are formed as a consequence of deamidation, isomerization, and racemization during trypsin digestion using buffer at pH 8. Normally, deamidation of Gln is much slower compared to the deamidation of Asn.

It is important to avoid inducing in-vitro deamidation during sample preparation while identifying in-vivo deamidation sites on proteins. Modified sample preparation procedures may be needed to identify deamidation modifications on proteins. For example, a protein sample can be subjected to trypsin digestions under pH 6.5 and pH 8, respectively. In-vivo deamidation products can be distinguished from in-vitro products by profiling the digested proteins (pH 6.5 vs. pH 8) by LC-MS. Peptides obtained from protein digestion at pH 6.5 serves as a control to filter in-vitro induced deamidation peptide products at pH 8.

### (d) Post-Translational Modifications

Post-translational modifications play an essential role in protein functions which regulate cellular process. Post-translational modifications occur after the translation of mRNA. It is a biochemical process where amino acid residues are covalently modified by removing or adding molecules in a protein. These modifications can change a protein's folding, biological function, immunogenicity, and/or stability (Farley et al., METHODS IN ENZYMOLOGY (2009) 463: 725-762; Walsh et al., NATURE BIOTECHNOLOGY (2006) 24(10): 1241-1252). Post-translational modifications include, but are not limited to, acetylation, acylation, γ-carboxylation, β-hydroxylation, disulfide bond formation, glycosylation, methylation, phosphorylation, proteolysis processing, and sulfation. Among these modifications, acetylation, methylation, amidation, phosphorylation, and glycosylation are commonly found in approved therapeutic protein drugs and candidates in discovery or clinical trial stages. Heterogeneous species can be formed after post-translational modifications, such as glycosylation or amidation, which may or may not alter protein folding and function. Thus, characterization of post-translational modifications provides structural insight to enable associating structure with biologic functions. Use of mass spectrometric methods for characterization of post-translational modifications on proteins, MS fragmentation techniques play critical roles in producing specific types of fragments to allow identifying post-translational modification sites in which amino acid residues are modified. Structural elucidation of amidation is described above in the section “C-Terminal Modification”. Characterization of glycosylation is illustrated in the following section “Higher-Order Structures”. Examples of characterization of proteins with methylation, acetylation, and phosphorylation modifications are demonstrated, respectively below.

### (i) Methylation and Acetylation

Methylation involves adding one or more methyl groups onto amino acids. For example, N-methylation can be found at the N-Terminal alanine, isoleucine, leucine, methionine, phenylalanine, proline, tyrsosine and/or the side chains of lysine, arginine, glutamine, asparagine or the imidazole ring of histidine residues (Paik et al., YONSEI MEDICAL JOURNAL (1986) 27(3): 159-177). O-methylation can be observed either at a C-Terminal cysteine, leucine, lysine or at the side chain of glutamic acid and aspartic acid residues (Paik (1986) supra). S-methylation can be noted at the side chains of methionine and/or cysteine residues. Acetylation transfers an acetyl group to the side chain of lysine (also known as lysine acetylation) or the N-terminal amino acid residue (also known as N-terminal acetylation). In general, methylation and acetylation modifications on proteins remain unchanged during sample preparation (for example, digestion by enzymes). After protein digestion, peptide fragments in solution including methylated and/or acetylated peptides are subjected to LC-MS analysis. Methylated peptide species can be identified by additional masses (for example, +14 Da for mono-methylation, +28 Da for di-methylation) followed by LC separation. Though tri-methylation and acetylation modifications provide the same additional mass of 42 (Da), the identification can be carried out by the CID-MS² fragmentation. For example, an unique immonium ion of m/z 126 can be observed in acetylated lysine but not present in tri-methylated lysine residues (Farley (2009) supra). In addition, a neutral loss of 59 Da, corresponding to the loss of trimethylamine, is unique for a tri-methylated lysine.

### (ii) Phosphorylation

Phosphorylation typically occurs at serine, threonine or tyrosine residues of proteins or peptides. In general, phosphorylation is a reversible post-translational modification occurring in cellular process to control protein activities. Phosphorylation is one of liable post-translational modifications. The phosphate groups on serine and threonine residues can compete with the peptide bones as preferable cleaved sites. Upon CID activation, peptides containing phosphorylated amino acid residues tend to lose the phosphor groups before they fragment along with the peptide backbone. As a result, mixed fragments are obtained, which cannot be differentiated between unmodified and phosphorylated peptides. To avoid obtaining ambiguous peptide sequences from phosphorylated proteins, a combination of CID (CID-MS²), ETD (ETD-MS²) and CRCID (CID-MS³) fragmentation techniques can be used to characterize phosphorylation modifications on proteins (Wu et al., JOURNAL OF PROTEOME RESEARCH (2007) 6: 4230-4244).

For example, after denaturation, reduction, and alkylation a phosphorylated protein sample is subjected to digestion using Lys-C to generate large proteolytic peptides. A large pore size of monolithic LC column such as polystyrene-divinylbenzene (PS-DVB, 50 μm i.d.×10 cm) can be used to separate large peptides including unmodified and phosphorylated peptides. CID and ETD are operated under both dependent and independent modes on a mass spectrometer (for example, Thermo LTQ Obitrap Elite ETD). Under an independent mode, use of CID and ETD can manually select less intensity of precursor ions (usually phosphorylated peptides) for subsequent fragmentation (for example CID-MS³), which is normally missed during data dependent experiments. Furthermore, fewer fragment ions (c and z ions) are obtained for a large peptide with a less charge (for example, +2) in the ETD-MS² scan, resulting in insufficient fragmentation for peptide assignment. A combination of using ETD (ETD-MS²) following CRCID (CID-MS³) via isolating an product ion produced in the ETD scan step can produce substantial c and z ion series along with phosphorylation sites on peptides. The peptide assignment for phosphorylated peptides and unmodified peptides is achieved using software(s) (for example, PepFinder and/or Proteome Discoverer). Besides mapping fragment ions, HPLC retention times and masses (for example, a loss of 98 Da as signature of phosphorylated peptide) are the key to assign the peptide identity.

### (e) Higher Order Structures

Higher order structures (HOS) of a biologic protein include the secondary, tertiary, and quaternary structures. HOS provide a three-dimensional (3D) confirmation, which plays an important role in its biological function. HOS are considered to be critical quality attributes because changes in HOS may affect efficacy or safety of biologic drugs. Characterizing HOS of a biologic protein is required by regulatory agencies (for example, USFDA quality by design, QbD and ICH Q5E (ICH HARMONISED TRIPARTITE GUIDELINE “Comparability of Biotechnological/Biological Products Subject to Changes in Their Manufacturin Process,” Q5E, Current Step 4 Version, dated Nov. 18, 2004)). HOS are often required during manufacturing of biologics (for example, comparability evaluations), formulation, stability assessment, and process development. Circular dichroism (CD) spectroscopy (Li et al., JOURNAL OF PHARMACEUTICAL SCIENCES (2011) 100(11): 4642-4654), X-ray crystallography (Harris et al., J. MOL. BIO. (1998) 275: 861-872), and nuclear magnetic resonance (NMR) (Amezcua et al., JOURNAL OF PHARMACEUTICAL SCIENCES (2013) 102(6): 1724-1733) are the conventional tools used to analyze HOS of a protein.

Hydrogen/deuterium exchange coupled with mass spectrometry (HDX MS) can be used to probe HOS of a biologic. Unlike CD spectroscopy, HDX MS can provide a 3D confirmation of an intact molecule (Engen, ANAL. CHEM. (2009) 81(19): 7870-7875) and a local confirmation of fragments of a biologic, such as peptides (for example, peptide epitopes) (Coales et al., RAPID COMM. MASS SPECTROM. (2009) 23: 639-647). For example, the exchange of protein backbone amide hydrogen with deuterium (CO—NH→CO-ND) provides conformational dynamics of the molecule in solution at a physiological pH (for example, pH 7.5, room temperature). After quenching a HD exchange reaction (for example, pH 2.5, 0° C.), a biologic protein is subjected to digestion (for example, pepsin digestion at pH 2.5) and digested peptides then are analyzed by MS. The HDX rate observed in peptides provides an indication of protein HOS, which can be measured based on the mass gain over time in solution via MS. For example, slow exchange occurs in regions buried in the core of a 3D structure and in heavily glycosylated peptides. Fast exchange often occurs in regions located on the surface of protein structure and in peptides with little or no glycosylation. Advantages of HDX MS over x-ray crystallography and NMR spectroscopy are: 1) that it provides dynamic conformational information of native biologics in solution; 2) that it is unlimited by the size of proteins or biologics being interrogated; and 3) sensitivity (i.e., less material required for HDX MS analysis) (Berkowitz et al., NATURE REVIEWS DRUG DISCOVERY (2012) 11: 527-540). HOS of biologics also include disulfide bonds, disulfide knots, and glycosylation, which are described in the following sections.

### (i) Disulfide Bonds

Disulfide bonds (—S—S—) primarily control the folding of three-dimensional protein structure, and generally fall into three groups: 1) intra-chain disulfide bonds; 2) inter-chain disulfide bonds; and 3) disulfide knots. In general, intra-chain disulfide bonds stabilize the tertiary structure and the inter-chain disulfide bonds involve in stabilizing quaternary structure. Disulfide knots can improve protein structural stability. Any modifications to the process of producing a biologic (e.g., changes in cell lines, cell culture medium, agitation force etc.) have the potential to cause protein conformational changes due to disulfide bond rearrangements (for example, unpaired or mispaired disulfide bonds). Thus, disulfide bonds are critical structural attributes, which need to be monitored for quality control purposes during manufacture or storage of biologic or biologic reference.

Intra-chain disulfide bonds occur within a single polypeptide whereas inter-chain disulfide bonds are formed between two polypeptide chains through oxidation of thio (—SH) groups on cysteine residues. A conventional approach for characterizing disulfide bonds includes comparing reduced and non-reduced peptide maps to help locate disulfide bonds on peptide backbones. A protein sample is digested by enzyme with and without reduction and alkylation to generate two protein digests (for example, protein digest 1 (PD1) with reduction and alkylation; protein digest 2 (PD2) without reduction and alkylation). PD1 and PD2 are subjected to LC-MS analysis. The disulfide-linking peptide (DSLP) can be found in the PD2 sample using a theoretical mass to locate retention time on LC-MS mass chromatogram. Then, the sequence of DSLP can be determined using ETD to cleave a disulfide bridge followed by CID to break peptide amide bonds subsequently. As expected, DSLP should not be detected in a PD1 sample. However, LC-MS analysis cannot differentiate an intra-chain disulfide bond from an inter-chain disulfide bond. The sample can be subjected to SDS-PAGE gel electrophoresis under reduction and non-reduction conditions. For a sample containing an intra-chain disulfide bond, no additional band can be discerned in both reducing and non-reducing gels. If it is an inter-chain disulfide bond, two additional bands (lower molecular weights) can be found in a SDS-PAGE gel run under reducing conditions.

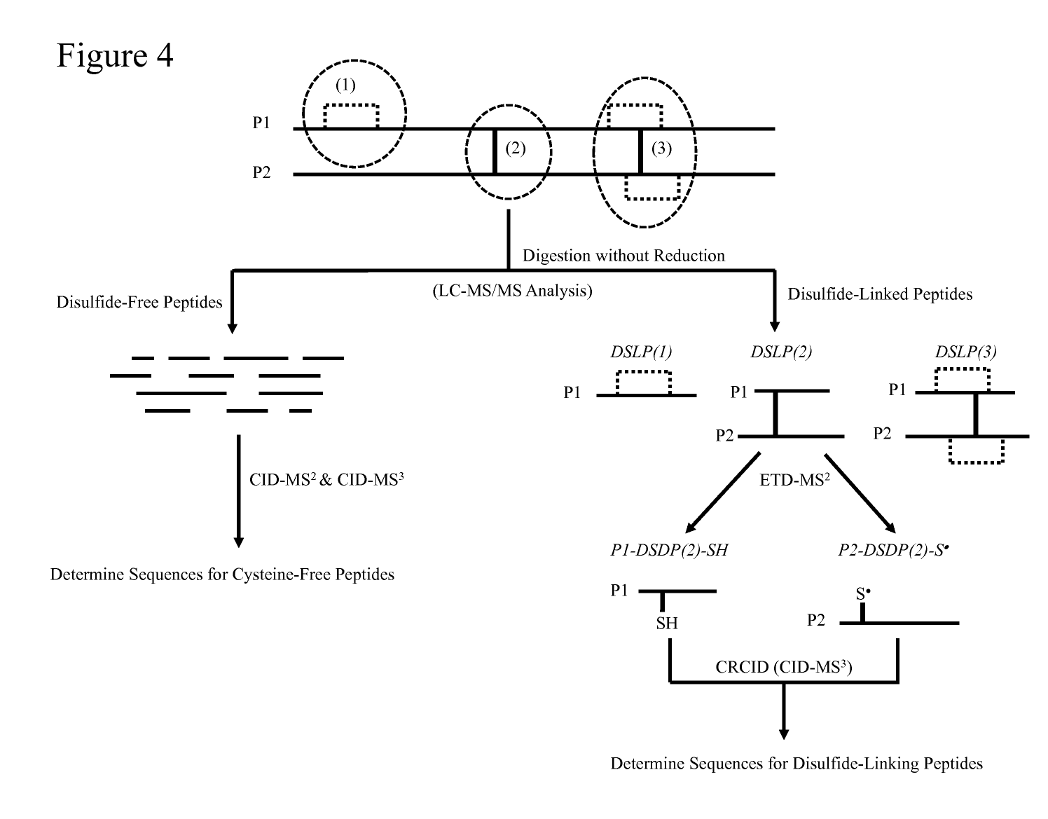

If a molecule of interest contains multiple disulfide bonds, the analysis of disulfide bonds is more complicated. Depending on the protein sequence and the locations of disulfide bonds, a strategy of using multiple enzymes and multi-fragmentation techniques to digest proteins into peptides containing only a single disulfide bond is ideal for mapping the disulfide bonds (Wu et al., ANAL. CHEM. (2009) 81(1):112-122); Wu et al., ANAL. CHEM. (2010) 82(12): 5296-5303). By way of example, and as shown in FIG. 4, an exemplary protein consisting of two polypeptides (P1 and P2) connected via two inter-chain disulfide bonds, where two intra-chain disulfide bonds are located in P1 and one intra-chain disulfide bond is placed in P2 can be characterized as follows. The protein without reduction is digested using multiple enzymes to produce 3 disulfide-linking peptides, referred to as DSLP(1), DSLP(2), DSLP(3), and many disulfide-free peptide fragments. Then, this protein digest (without reduction) is subjected to LC-MS/MS analysis through a reverse-phase HPLC separation (for example, use of Agilent Zorbax 300SB-C18 column, 5 μm particle size, 2.1 mm id×15 cm) coupled with a mass spectrometer (LTQ Obitrap Elite ETD). Again, theoretical masses of three predicted DSLPs are monitored to find their corresponding LC-MS chromatograms. Once the three DSLPs are located, the disulfide bonds can be cleaved using ETD (ETD-MS²) on DSLPs to produce disulfide dissociated peptides (DSDPs). For example, DSLP(2) is broken into two disulfide dissociated peptide species: P1-DSDP(2)-SH and P2-DSDP(2)-S* (see FIG. 4), which are further fragmented to yield c and z ions using CRCID followed by ETD-MS², and to produce b and y ions using CID-MS³ followed by CID-MS². The disulfide-free peptides are fragmented by CID-MS² and/or CID-MS³.

The assignment of DSLPs is based on the assumption that the locations of disulfide bonds on the polypeptide backbones (P1 and P2) are predictive of the sequences for DSDPs. Hence, The fragmentation spectra of P1-DSDP(2)-SH and P2-DSDP(2)-S* including CID-induced b and y ions and ETD-induced c and z ions are used to search against theoretical fragmentations of this given protein using software(s) such as PepFinder and Proteome Discoverer. The sequences of disulfide-free peptides are assigned in a similar manner.

To assure the assignment of DSLPs (for example, P1 or P2 peptide fragments), the protein sample is subjected to a SDS-PAGE gel electrophoresis under reducing conditions. Two separated gel bands corresponding to polypeptides P1 and P2, respectively, are cut from the gel and digested with multiple enzymes. The extracted peptides from each gel piece are analyzed by LC-MS/MS. The sequences of polypeptides P1 and P2 then are determined using CID-MS² and/or CID-MS³.

If predicted DSLPs cannot be found, disulfide bond rearrangement may have occurred in the protein. To verify the absence of predicted DSLPs, the protein can be digested using the same multiple enzymes with reduction and subjected to peptide sequencing by LC-MS/MS methods as described above. Through peptide mapping and sequencing, the cysteine residues can be located on the peptide backbones. Then, the protein can be subjected to digestion using different multiple enzymes with and without reduction. This assumes that the disulfide bridges are shuffled to bear unexpected DSLPs such as unpaired or mispaired DSLPs. Thus, the use of different multiple enzymes can recover re-arranged DSLP fragments. These un-paired or mispaired DSLPs can be characterized and identified by LC-MS methods as described above.

### (ii) Disulfide Knots

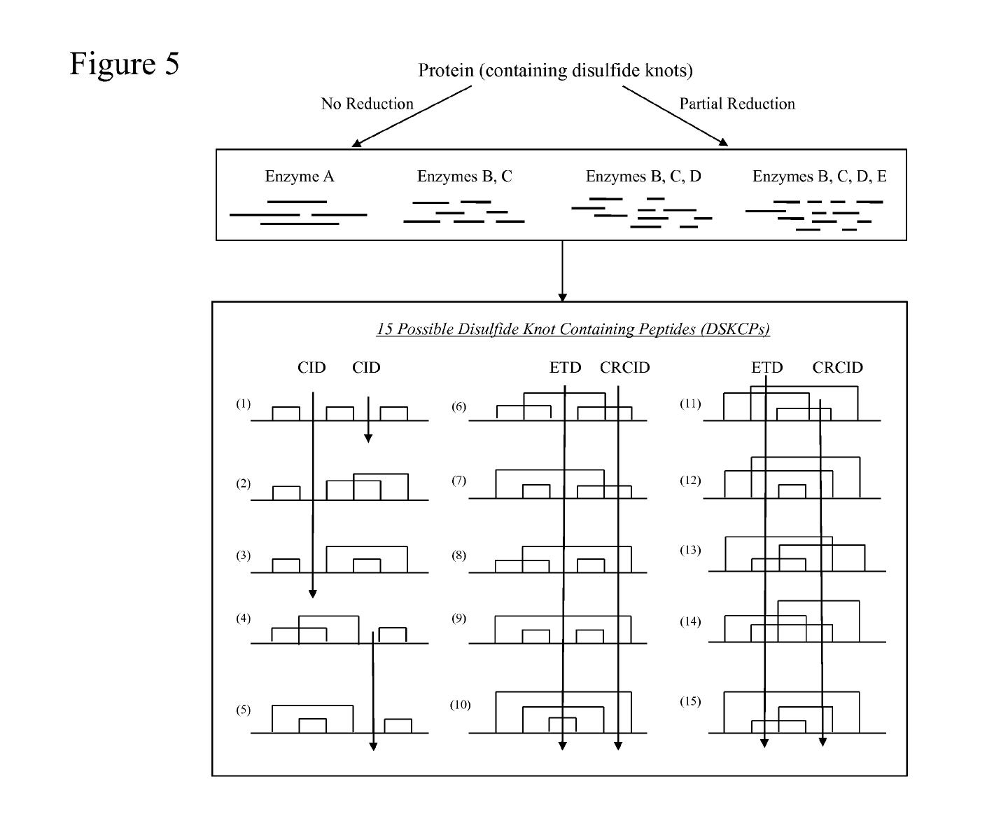

Disulfide knots are structural motifs often found in proteins and typically comprise at least three disulfide bonds (six cysteine residues), where one disulfide bond passes through the ring of the other two disulfide bonds. Some therapeutic protein biologics (for example, recombinant human arylsulfatase A) contain disulfide knots, which can be scrambled or shuffled during expression, purification, or storage. It can be difficult to verify a protein bearing disulfide knots with a correct position since there are many ways to arrange a disulfide knot (Ni et al., J. AM. SOC. MASS SPECTROM. (2012) 24: 125-133). Enzymes or CID typically do not cut the peptide backbone disposed within a disulfide knot. The generation of desirable sizes of peptide fragments is important in the successful characterization of biologics containing disulfide knots. Thus, a process of using staggered multi-enzymes and multiple fragmentation techniques has been developed, as is shown schematically in FIG. 5.

As shown in FIG. 5, a sample is subjected to digestion without reduction and/or with partial reduction using a series of staggered multiple enzymes. Enzyme A can be a single enzyme such as pepsin to produce fragments under an acidic condition. It is known that disulfide scrambling can take place under alkaline conditions. Thus pepsin digestion (pH 2) can avoid disulfide shuffling and serve as a negative control for scrambled disulfide bonds. Nevertheless, limited or large peptide fragments are produced by pepsin digestion due to the presence of disulfide knots. Enzymes B and C (for example, trypsin and Lys-C), B, C, D (for example, trypsin, Lys-C, Asp-N), and possibly B, C, D, and E (for example, trypsin, Lys-C, Asp-C, and PNGase F if there is glycosylation on a protein) can yield different, small sizes of fragments. The multiple enzyme digestions can be carried out at pH 6.8 and pH 8 to monitor false disulfide knots if induced during sample process. Yet, kinetically favorable mismatched disulfide bridge(s) can be formed from two adjacent cysteines during enzymatic digestion. To avoid artifacts associated with the breakage of cysteine knots introduced during sample processing, a method of partial reduction (for example, a protein sample reduced by TCEP in 6M guanidine hydrochloride/sodium acetate buffer, pH 4.6, at 37° C. for no more than 20 minutes) and alkylation (for example, a partially reduced protein sample incubated with NEM at 37° C. for 60 minutes in the dark) can be used to block the formation of S—S bonds from two adjacent cysteines prior to the enzymatic digestion (see FIG. 5). Then, all the digested samples are analyzed by LC-MS/MS using CID (CID-MS²), ETD (ETD-MS²), and CRCID (CID-MS³) to generate disulfide free fragments and disulfide knot-containing fragments, as shown in FIG. 5.

Although the use of theoretical masses can locate disulfide knot containing fragments on a LC-MS chromatogram, it can be difficult to map fragmentation spectra to confirm the correct sequence defining a disulfide knot from multiple possible structural arrangements (for example, the 15 possible arrangements shown in FIG. 5). Use of multi-tier fragmentation techniques (CID→ETD→ETD/CID-MS³) can simplify the structural assignment for disulfide knot containing peptides (Ni (2012) supra). For example, to identify a correct disulfide knot position from 15 possible structural arrangements having the same precursor mass shown in FIG. 5, CID (orange line) is used as the first tier to exclude the other ten possible structure rearrangements (for example, DSKCPs 6-15, FIG. 5). Only dissociated disulfide containing peptides can be observed for DSKCPs (1-5) using CID alone (FIG. 5). ETD (green line) serves as the second tier to fragment DSKCPs 6-15 if no dissociated disulfide containing peptides are observed after CID fragmentation in the first step. During this ETD step, disulfide knots are opened via cleavage of S—S bonds as illustrated in DSKCPs 6-15 (FIG. 5). Then, the use of CID-MS³ followed by ETD (i.e., CRCID; blue lines) generates more fragment ions for the assignment of DSKCPs. DSCKP fragments obtained from protein digests without reduction (for example, pepsin, pH 2; trypsin, pH 6.5 and pH 8) and/or with partial reduction (for example, TCEP/NEM, pH 4.6) prior to digestion are carried out to assure the assignment of cysteine knots reflected from a native biologic.

(iii) Glycosylation

Glycosylation is also important for the production of biologics because, for example, more than 90% of the protein drugs such as monoclonal antibodies are glycoproteins. Furthermore, glycosylation is the most complex post-translational modification, where sugar moieties play roles in protein binding, conformation, stability, and activity (Walsh (2006) supra). Glycosylation can significantly impact on the potency, pharmacokinetic, or immunogenicity of a biological drug if any modifications (for example, changing cell lines) occur during the manufacturing process. Additionally, it can be difficult and impractical to produce a homogeneously glycosylated protein. Although the production of biologics is monitored under a good manufacturing process (GMP), heterogeneous species of glycoproteins (for example, different forms of glycan linked with a protein) can only be minimized. Thus, glycosylation is a critical attribute for a therapeutic protein.

Based on the glycosidic linkage between protein and glycan, glycosylation can be grouped into five types: N-linked (glycan attached to the amino group of asparagine), 0-linked (glycan bound to the hydroxyl group of serine or threonine), C-linked (glycan added onto the indole ring of tryptophan), phospho-linked (glycan linked to serine through phosphodiester bond), and glypiation (glycosylphosphatidylinositol anchor linked a phospholipid and a protein through a glycan linkage) (Ni (2012) supra). Among these five glycosylation types, the most common types are N-linked and O-linked. Characterization of glycosylation involves four steps: 1) glycan removal (known as deglycosylation); 2) glycosylation site determination; 3) peptide sequencing; and 4) glycan analysis. Deglycosylation is essential for identification of the peptide and the site of glycosylation. After producing the peptide backbone via deglycosylation, glycan attached on the peptide (known as glycopeptide) can be predicted by subtracting the molecular weight of the peptide from that of the glycopeptide.

Based on the type of glycosylation, various approaches can be used to remove glycans from a glycoprotein. For example, PNGase F can remove most N-linked glycans except for a fucose-all-3) bound to the Asn-GlcNAc linkage. N-glycosidase A can be used to release oligosaccharides containing an α(1-3) fucose core. There is no enzyme like PNGase F that can remove “intact” O-linked glycans. Rather, the removal of O-linked glycans can be achieved using a series of exoglycosidases to hydrolyze various types of monosaccharides until only the Gal-β(1,3)-GalNAc core remains. O-glycosidase (endo-α-N-acetylgalactosamindase) can then release the Gal-β(1,3)-GalNAc core structure from the serine or threonine residues (Iwase et al., METHODS IN MOLECULAR BIOLOGY (1993) 14: 151-159). Determination of glycosylation site can be accomplished in parallel with peptide sequencing because N-linked asparagine or O-linked serine/threonine residues are the known as glycosylation sites. For glycan analysis, glycan can be collected after enzymatic or chemical digestion. The methods for characterization of N-linked and O-linked glycosylation are described below.

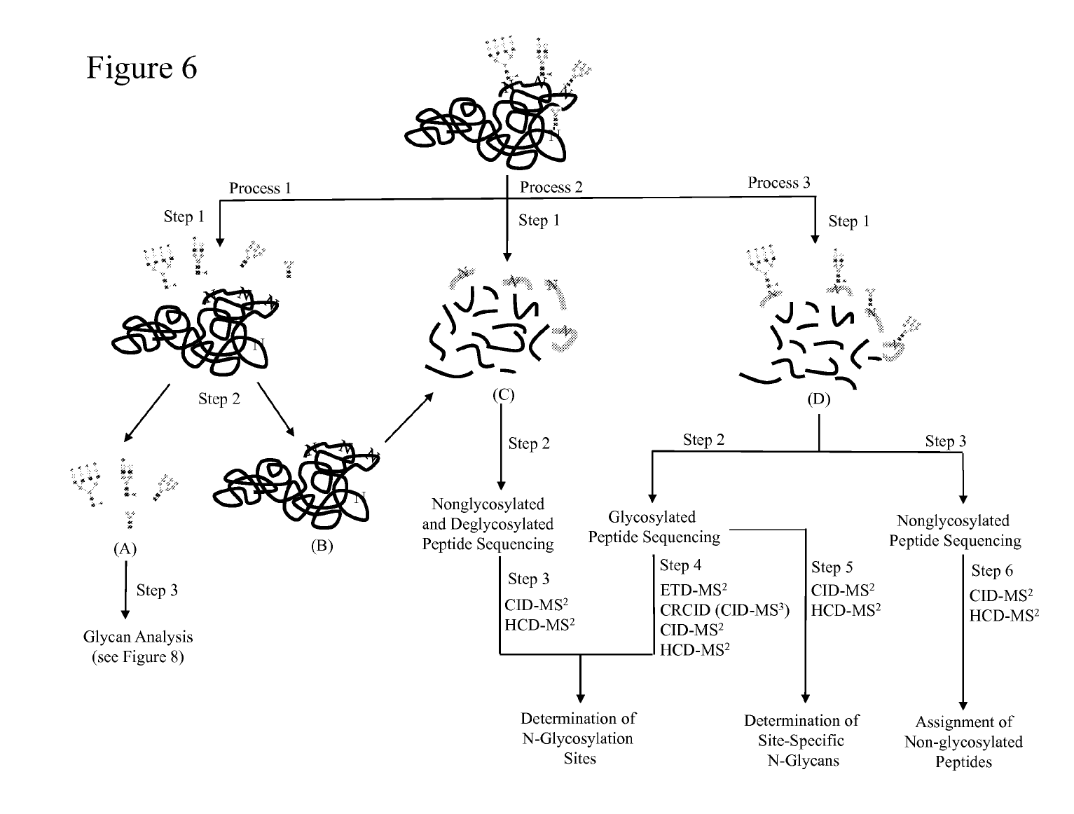

As shown in FIG. 6, N-linked glycosylation can be determined via three integrated processes: 1) deglycosylation (process 1); 2) multi-enzymatic digestion including deglycosylation (process 2); and 3) multi-enzymatic digestion without deglycosylation (process 3). For example, a glycoprotein sample is subjected to denaturation (6M guanidine hydrochloride, room temperature), reduction (200 mM DTT at 37° C. for 30 min), and alkylation (200 mM IAA at room temperature for 45 min in dark) prior to each of process 1, 2, and 3. Denaturation and reduction opens the three-dimensional glycoprotein structure, thus permitting an enzyme accessing the glycosylation and/or proteolytic sites. In process 1, step 1, the denatured and reduced glycoprotein sample is subjected to deglycosylation using PNGase F to generate detached N-linked glycans and an intact protein. In step 2, the detached N-linked glycans then are isolated from the intact protein using 10,000 Da molecular weight cut off filter to produce a glycan fraction (Fraction A) and a protein fraction (Fraction B). The glycan fraction (Fraction A) is collected for glycan analysis by LC-MS/MS using CID-MS² and CID-MS³.

Process 2 involves the use of multiple enzymes including PNGase F (see step 1) to generate a mixture of non-glycosylated and deglycosylated peptides (Fraction C). The intact protein fractionation (Fraction B) obtained in Process 1 (Step 2) can also be used to generate fractionation (Fraction C) by adding the same multiple enzymes without PNGase F as described in Process 2 (Step 1). The resulting fraction (Fraction C) then is subjected to LC-MS/MS analysis (Step 2) using CID-MS² and/or HCD-MS² (FIG. 6, Process 2/Step 3).

Process 3 produces non-glycosylated and glycosylated peptides (Fraction D) using the same multiple enzyme(s) as process 2 except no PNGase F (step 1). ETD can preserve labile post-translational modifications (PTMs) so as to preserve glycans attached on a peptide backbone. Alternatively, CID and/or HCD can generate fragment ions predominantly from cleavage of glycosidic bonds on glycans without breaking the peptide amide bonds (Wu et al. (2007) supra; Ye et al., ANAL. CHEM. (2013) 85(3): 1531-1539; Miller et al., J. PHARM. SCI. (2011) 100(7): 2543-2550). Hence, glycosylation sites can be identified using CID, HCD and ETD. For example, glycosylated (N-linked) and nonglycosylated peptides (Fraction D) are subjected to LC-MS/MS analysis. ETD (ETD-MS²), CRCID (CID-MS³), CID (CID-MS²) and/or HCD (HCD-MS²) are used to fragment glycosylated peptides to determine N-glycosylation sites (FIG. 6, Process 3/Step 4). CID (CID-MS²) and/or HCD (HCD-MS²) are applied to glycosylated peptides, which enable determining site-specific glycans (Process 3/Step 5). Matching observed glycopeptide masses with theoretical masses of glycans and peptides indicate if the protein is properly glycosylated.

Nonglycosylated peptides present in Fraction D can be distinguished from glycosylated peptides once N-linked peptides are identified. The sequence of nonglycosylated peptides can be obtained using CID-MS² and/or HCD-MS² (Process 3/Step 6). Through mapping of deglycosylated peptides (Process 2/Step 3), nonglycosylated peptides (Process 2/Step 2 and Process 3/Step 3), and glycosylated peptides (Process 3/Step 2), the N-linked glycosylation sites can be determined

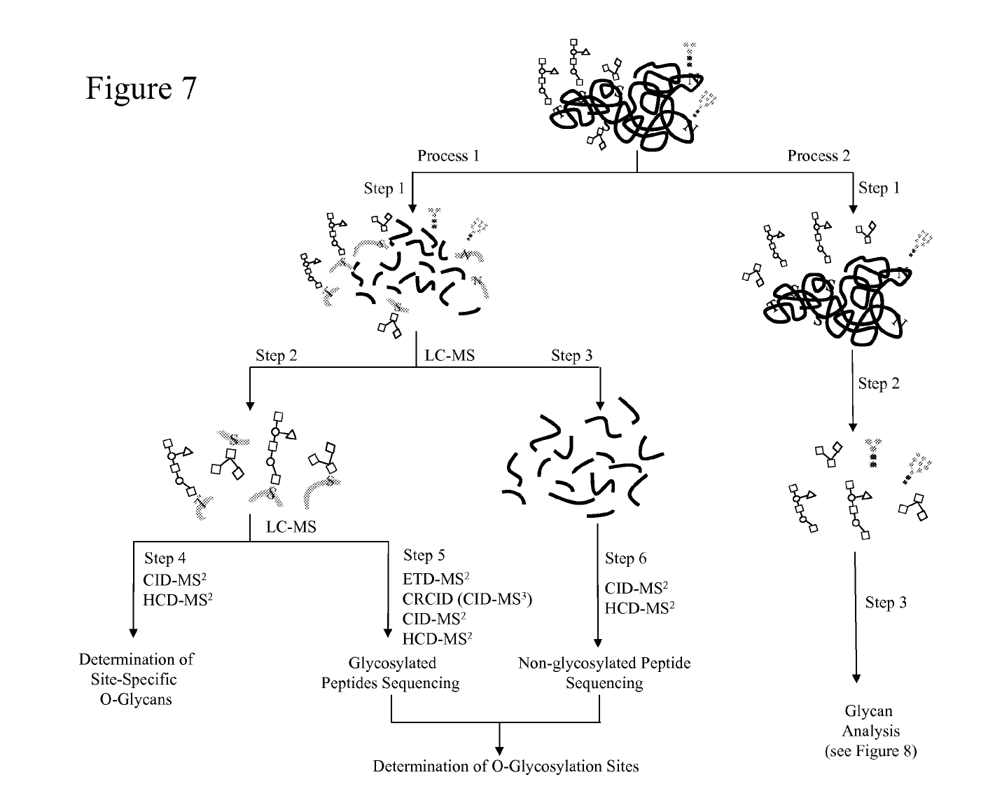

The characterization of O-linked glycosylated biologics is more complicated because there is no single enzyme available to cleave an “intact” O-linked glycan complex from serine or threonine residues on the protein. Traditionally, O-glycans are released through β-elimination using strong bases such as sodium hydroxide or hydrazinolysis using hydrazine (Patel et al., BIOCHEMISTRY (1993) 32: 679-693). Although, β-elimination reaction can remove an intact O-glycan, the protein can be degraded under alkaline conditions. In addition, O-linked glycoproteins may contain N-linked glycans. Therefore, as shown in FIG. 7, the characterization method for N-linked glycosylation can be modified to enable O-linked analysis, and involves two processes: an enzymatic process (Process 1) and a chemical and enzymatic process (Process 2).

As shown in FIG. 7, the glycoprotein sample is subjected to denaturation, reduction, and alkylation prior to Process 1 and Process 2. In Process 1 (Step 1), the reduced glycoprotein is digested with multiple enzymes including PNGase F to generate O-linked glycopeptides, deglycosylated and non-glycosylated peptides. Then the digest sample is subjected to LC-MS analysis. Using the predicted masses of known and possible O-linked glycopeptides, they can be measured under a selected ion monitoring (SIM) mode on a mass spectrometer (Step 2). Once detecting the masses matched with predicted O-linked glycopeptides, O-linked glycans (Step 4) can be fragmented using CID (CID-MS²) and/or HCD (HCD-MS²) to determine site-specific O-glycans. O-glycopeptides are sequenced using ETD (ETD-MS²), CRCID (CID-MS³), CID (CID-MS²), or HCD (HCD-MS²) (Process 1/Step 5). Non-glycosylated peptides can be located on the LC-MS chromatogram based on their predicted masses (Step 3). Non-glycosylated peptide sequencing is carried out using CID (CID-MS²) and HCD (HCD-MS²) (Step 6). Through mapping of glycosylated and non-glycosylated peptides (Step 5 and Step 6)), O-linked glycosylation sites are determined

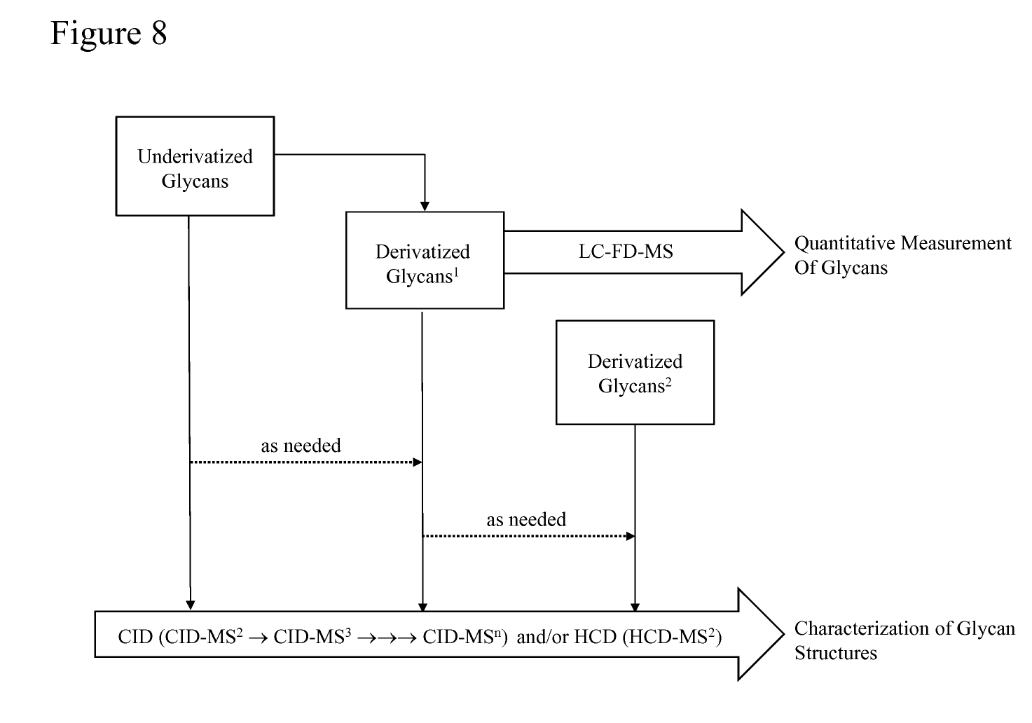

Process 2 of FIG. 7 can be used for glycan analysis. Chemicals (for example, GlycoProfile™ β-Elimination Kit, Sigma) can be used to free glycans (both O- and N-linked glycans) (Step 1) from the glycoprotein. The released glycans then are separated from an intact protein using 10,000 Da molecular weight cut off filter (Step 2). The resulting, harvested glycans are subsequently subjected to glycan analysis (Step 3). The analysis of the glycans can be important in the characterization of the biologic because the heterogeneity of glycoprotein is mainly due to glycan content, which can differ by sequence, chain length, branching site, and position of linkage to the peptide chain. For example, there are four different N-linkages to asparagine (Asn) residues on N-linked glycoproteins: 1) N-acetylglucosamine (GlcNAc); 2) N-acetylgalactose (GalNAc); 3) glucose; 4) rhamnose. N-glycans are usually attached to a protein at Asn-X-Ser or Asn-X-Thr sequences, where X can be any amino acid except Pro. The most common N-linkage is GlcNAc-Asn, consisting of three general types of N-glycans: oligomannose, hybrid, and complex (Kornfeld et al., ANN. REV. BIOCHEM. (1985) 54: 631-664). Importantly, glycan analysis is always included as part of quality control for glycoprotein drug products since sugar moieties involve in protein folding, stability, and biological functions. Glycan analysis can provide qualitative (i.e., characterization of glycan structures, such as glycan type including sequence, chain length, position of linkage) and quantitative (for example, relative amounts of each glycan type) measurement (see, FIG. 8).

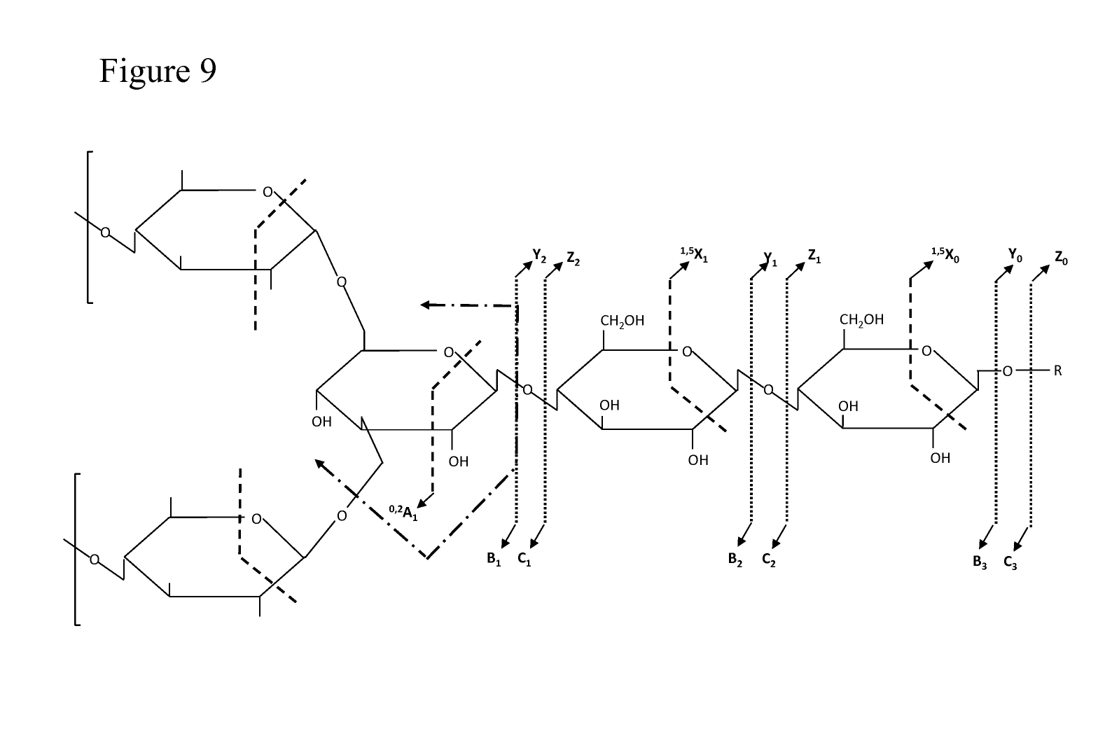

Analysis of the glycan moieties attached to a protein can be performed through enzymatic or chemical processes (referred to deglycosylation) as illustrated in FIG. 6 (Process 1, Step 1) and FIG. 7 (Process 2, Step 1). For example, following the characterization method for N-linked glycosylated proteins (FIG. 6, Process 1: Step 1→Step 2→Step 3), the released N-glycan sample is analyzed by LC-MS using a HILIC LC column (for example, Thermo GlycanPac AXH, 1.9 μm, 2.1 mm id×15 cm) through a linear gradient from high to less organic phases (for example, from 80/20 to 60/40 acetonitrile/water in 40 minutes) at a flow rate of 200 μL/min. Each type of glycan molecule is quantitatively measured under a selected ion monitoring mode (SIM) on a mass spectrometer according to their predicted masses. CID (CID-MS²) fragmentation then is used to fragment each selected glycan ion, thus permitting to assign the glycan structure. For a comparability study (for example, two different lots of biologic drugs are produced in manufacture, Lot 1 vs. Lot 2), the released glycans from Lot 1 and Lot 2 are profiled by LC-MS. Through LC-MS profiling, most glycan species can be distinguished between Lot 1 and Lot 2 based on the LC separation and their masses. If glycan isomers cannot be separated by LC; the MS/MS experiments using CID-MS² and/or CID-MS³ can further break their molecules to produce unique fragmentation ions (product ions). Use of a combination of theoretic masses and fragmentation spectra to search against a glycan database (for example, a glycan library built using Glycoworkbench) can assign glycan identities. Nevertheless, some of the glycans may be difficult to measure quantitatively because of poor ionization of glycan structures by MS, or identify because of the complex isomeric nature of glycans. Labeling glycans via derivatization can be used to improve the detection of glycans (Ruhaak et al., ANAL. BIOANAL. CHEM. (2010) 397: 3457-3481). Depending upon the complexity of glycan structures, a multi-tier strategy can be applied for quantitative and qualitative glycan analyses (see, FIG. 8). For example, released glycoforms are labeled with fluorescent dye such as 2-aminobenzamide (2-AB) to create derivatized glycans (denoted as Derivatized Glycans¹, FIG. 8) through reductive amination. Then, 2-AB labeled glycans are quantitatively analyzed by LC-FD-MS. In order to assign glycan structures, a multi-stage approach is used as illustrated in FIG. 8. For example, LC-MS/MS using CID (CID-MS², CID-MS³, and possibly CID-MSⁿ) and/or HCD (HCD-MS²) are used to characterize underivatized glycans as the first-stage. Based on the glycans of interests, some glycans may need to be derivatized. For example, the reducing end of branching glycans can be labeled for example, labeled with 2-AB and then permethylated to provide another set of derivatized glycans (denoted as Derivatized Glycans², FIG. 8). Permethylation of glycans replaces hydrogens on hydroxyl, amine, and carboxyl groups with methyl groups. Then CID-MS² or HCD-MS² can be used to break glycosidic bonds to generate fragment B, C, Y, Z ions (FIG. 9, blue dotted lines) and to cleave cross-ring bonds to produce fragment A and X ions (FIG. 9, red dashed lines) (Domon et al., GLYCOCONJUGATE JOURNAL (1988) 5: 397-409). Generally, permethylated glycans are analyzed by MS under a positive ionization mode. By monitoring predicted masses of permethylated glycans as precursor ions, methylated glycans are subsequently fragmented by CID (CID-MS², CID-MS³, and possibly MSⁿ). Because methylated functional groups (for example, hydroxyl, amine, and/or carboxyl groups) noted on glycans cannot be the linkage sites, this permits the assignment of branching linkage position(s). Additionally, D ions can be generated by B and Y cleavages of two glycosidic bonds adjacent to the monosaccharide unit at the branch (FIG. 9, green dotted and dashed lines) under a negative ionization mode by MS (Harvey, MASS SPCETROMETRY REVIEWS (1999) 18: 349-451). The use of fragmentation patterns (for example, A, B, C, X, Y, Z, and D ions) and derivatization method(s) (for example, 2-AB labeling or permethylation) can permit the assignment of branching glycan structures.

### (II) Characterization of Reference Biologic Following Stress Testing

Once the reference biologic has been characterized, the reference biologic then is analyzed following exposure to one or more stress conditions to identify areas susceptible to modification or degradation. The instability of biologic drugs can result from chemical or physical degradation. Chemical degradation (for example, deamination, oxidation, isomerization, disulfide bond rearrangement, proteolysis, racemization, and β-elimination) involves alternations in molecular structure via bond cleavage or formation. Physical degradation (for example, denaturation, adsorption, aggregation, fragmentation, and precipitation) involves changes in the secondary, tertiary, or quaternary structure of a protein. According to ICH guidelines (Q5C), stability testing needs to support the shelf life of biologic drugs by considering any conditions affecting potency, purity, and quality (Ganan Jimenez et al., Presentation at the EUROPEAN MEDICINES AGENCY'S ICGH CGC ASEAN training in Kuala Lumpur (May 30-31, 2011)). Thus, testing is conducted by exposing the reference biologic under stress conditions to investigate their impact, if any, on structure and/or biological function of the biologic. Various characteristics of the as-stressed reference biologic can be analyzed by mass spectrometric analysis to determine any changes or modifications in biologics after exposure to stress conditions. These characteristics used for determination of protein stability serve as quality attributes for the biologic molecules Stress conditions commonly used for the stability testing include temperature, pH, light, ionic strength, mechanical stress, proteolysis (enzymatic degradation), formulation, peroxide contamination, antimicrobial preservatives, leachable, and accelerated aging, each of which are discussed below.

### (1) Stress Conditions

### (a) Temperature

In general, the effect of temperature on the biologics can be determined under two sets of conditions: 1) thermal stress whereby heat is applied to elevate the ambient temperature of the sample; and 2) storage temperature, which contributes to the shelf life of the biologic. A thermal stress condition typically is conducted during accelerated stability testing to induce potential protein degradation products in a short period time. The major damaged protein products usually are protein aggregates. To ensure shelf life, the storage temperature needs to be determined for each biologic drug because improper storage temperatures can cause degradation. For example, deamidation may occur to a protein if it is stored in amine buffers under inappropriate temperatures (Patal et al. (2011) supra). Because amine buffers (e.g., Tris and histidine) have high temperature coefficients, storage temperatures that are different from the temperature of preparation can shift formulation pH thus resulting in deamidation during storage. In addition, solution conditions, for example, temperature, protein concentration, pH, and ionic strength may lead to protein aggregation (Ganan Jimenez (2011) supra). By altering solution conditions, such as temperature, soluble aggregates are usually reversible. As a result of these studies, the appropriate storage conditions can be chosen.

(b) pH

Protein degradation can occur when the pH of a solution changes, which can take place during production (for example, cell culture and purification), formulation, storage, or sample processing (for example, solution digestion). Deamidation, isomerization, racemization, oxidation, hydrolysis, aggregation, and truncation are typical outcomes when changes in pH occur. For example, deamidation is a base-catalyzed reaction in the pH range 5 to 8, whereas isomerization is an acid-catalyzed reaction in the pH range 4 to 6. In other words, deamidation of asparagine residues can be accelerated under neutral and alkaline conditions and isomerization of aspartic acid can be induced under mild acidic conditions. Thus, asparagine deamidation and aspartic acid isomerization are a major stability concern during formulation of biologics (Wakankar et al., J. PHARM. SCI. (2006) 95(11): 2321-2336).

Racemization can be observed at alkaline pH, for example, as consequences of asparagine deamidation. Furthermore, pH can effect cysteine oxidation, which occurs at alkaline pH. Once cysteine oxidation occurs the disulfide bonds can be rearranged leading to mispaired disulfide bonds or scrambled disulfide bridges, which can change protein folding or result in protein aggregation (Li (1995) supra). On the other hand, if any shift to lower pH, hydrolysis and truncation can be induced. A common example of this occurs during antibody hydrolysis, which can take place in the hinge region if the pH is altered from 9 to 5 or outside of hinge region under more acidic conditions such as pH<4 (Gaza-Bulseco et al., PHARMACEUTICAL RESEARCH (2008) 25(8): 1881-1890).

### (c) Light

Light can trigger oxidation in the presence of oxygen, refer to photo-oxidation. Photo-oxidation normally involves side chains of tryptophan, tyrosine, histidine, methionine, and cysteine residues during production and storage of protein biologics. A protein molecule, excited by absorbing certain wavelength of light, can convert oxygen molecules to reactive singlet oxygen atoms, resulting in modifications in amino acid residues. Photo-oxidation can cause a change in protein structure, stability, or immunogenicity (Kerwin et al., J. PHARM. SCI. (2007) 96(6): 1468-1479). For example, aggregation can be observed in a protein due to cross-linking of oxidized tyrosine species such as mono-, di-, tri-, and tetra-hydroxyl tyrosines (Zhang et al., AAPS PHARMSCITECH (2007) 8(4): E1-E8). A protein itself can also react directly with another protein in a photo-energized manner via methionine or tryptophan residues at low pH, leading to aggregation (Li (1995) supra). Moreover, external catalysts such as formulation components, excipients, leachable, and metals etc. can affect the rate of photo-oxidation. To ensure the stability of biologic drugs, a photo-stability is usually performed using a light source to produce an output similar to the D65/ID65 emission standard (e.g., a daylight fluorescent lamp with visible and UV light) according to the guideline described in ICH Q1B (Zhang (2007) supra).

### (d) Ionic Strength

Ionic strength refers to the concentration of molecular ions, which are charged in solution. These molecular ions can be buffer salts, formulation components or proteins. A change in ionic strength in solution can cause structural instability during cell culture (e.g., change buffer medium), purification (e.g., switching solvents for ion exchange chromatography), formulation (e.g., modifying formulation components), and handling of proteins (e.g., dissolving high concentrations of protein in solution) (Patal (2011) supra). Deamidation, aggregation, truncation, and fragmentation typically are observed in biologics if ionic strength is altered in solution. It should be noted that ionic strength usually couples with solution conditions such as pH and temperature. This is why it affects deamidation rate of asparagine if changing formulation pH or increasing temperature in solution.

### (e) Mechanical Stress

Mechanical stress on biologic molecules includes agitation (for example, stirring, shaking, shearing, or pumping) as well as freezing and thawing. Aggregation is a common problem encountered during manufacture and storage of biologics.

There are different types of aggregations depending on the cause. Aggregation can result from protein undergoing a chemical degradation such as oxidation and deamidation. Mechanical stress induced aggregation can occur when the biologic is present in the interface between liquid and air, liquid and solid, or liquid and liquid. Freezing and thawing (F/T) can also promote aggregation in solution. Generally, F/T aggregates are reversible in solution. initially. Once undergoing multiple F/T cycles, water and ice crystals can produce a salting out effect leading to irreversible protein aggregates in solution. Aggregates are undesirable because they may cause immunogenicity when administered to a subject (Cromwell et al., THE AAPS JOURNAL (2006) 8(3): E572-E579).

### (f) Proteolysis

Proteolysis is a breakdown of proteins into peptides or amino acids via hydrolysis of peptide bonds by a protease. During the production of biologics, it is common to observe enzymatic degradation of protein molecules caused by protease contaminants in cell lines or residual proteases from the purification steps.

### (g) Formulation

During formulation of biologic drugs, the drugs are exposed to different stress conditions that may compromise biologic stability and quality. (Shire, CURRENT OPINION IN BIOTECHNOLOGY (2009) 20: 708-714). For example, aggregation is an issue for intravenous formulations of high dose biologic drugs, such as monoclonal antibodies. Biologics may be subjected to high concentrations of components used in a formulation to assess whether those components, either alone or in combination, cause the formation of aggregates. For example, sucrose included in a formulation can enhance antibody aggregation over time during storage due to protein glycation (Banks et al., J. PHARM. SCI. (2009) 98(12): 4501-4510), and formulations containing phosphate buffer can accelerate the rate of methionine oxidation. In addition, protein biologics can fragment if formulated under inappropriate buffering conditions (e.g., incorrect pH range).

### (h) Peroxide Contamination

Peroxide-induced oxidation can take place during the manufacturing of biologics. The source of peroxide usually can be found in formulation components such as polysorbate or polyethylene glycol, which are commonly used as pharmaceutical excipients. Peroxide-induced oxidation can also occur during storage of biologics if inappropriate containers are used (Patal (2011) supra). This is because peroxide can leach from plastic or elastomeric materials used in storage containers or container-closure systems including prefilled syringes.

### (i) Antimicrobial Preservative

Antimicrobial preservatives are included in formulation to increase shelf life of biologic products. During formulation development, the selection of antimicrobial preservatives should be included in the stress tests discussed here because certain preservatives (for example, benzyl alcohol) can induce protein aggregation (for example, rhGCSF) (Thirumangalathu et al., J. PHARM. SCI. (2006) 95(7): 1480-1497). The concentration of antimicrobial preservatives contained in formulation generally is specific for each biologic product. For example, phenol and m-cresol are common antimicrobial preservatives for biologic products. It is not always helpful to use high concentrations of phenol and m-cresol, which tend to increase the hydrophobicity of a formulation and form soluble and insoluble aggregates during storage over time.

### (j) Leachables

Undesired degradation products, induced by foreign components can be noted during manufacture, storage, and use of biologic products. According to the ICH guidance, drug substances stored in containers that properly represent actual containers used during manufacture should be subjected to stability testing, ICH Q5C (Ganan Jimenez (2011) supra). Any container closure systems used for storage or dosage use of biologics should be included in stability testing, ICH Q1A (R2) (“Guidance for Industry. Q1A(R2) Stability Testing of New Drug Substances and Products” U.S. Department of Health and Human Services, Food and Drug Administration (November 2003)). In most cases, leachables are trace metals in stainless steel containers used for production of biologics. Organic leachable can be from elastomers or glass used to store biologic products, or plastizers such as di-(2-ethylhexyl) phthalate (DEHP) used as infusion components during administration of drugs. Leachable-induced degradation mainly involves protein oxidation and aggregation, which possibly resulting in a loss of biological activity or an immunogenic issue.

### (k) Accelerated Aging

Accelerated testing provides supportive data to establish the shelf life of biologics. This stress is normally carried out beyond real time and real conditions to allow reveal degradation products, which can serve as stability indicators to determine the quality of biologic drugs during manufacture and storage (Ganan Jimenez (2011) supra). The accelerated aging conditions can be widely various depending on biologics. Temperature, pH, light, shaking, and freezing/thawing are typically stress parameters to be included for accelerated aging testing. If a room storage temperature (for example, 30° C.±2° C.) is intended for the biologic product, then the full-term stability studies (for example, 12 months) should be conducted at 30° C. Accelerated stability should be conducted at an elevated temperature (for example, 40° C.±2° C.) up to 6 months. The short-term accelerated data are useful in predicting longer-term shelf life of biologics and representing accidental exposures to other conditions

### (2) Determination of the Effect of Stress on the Structure and Function of the Reference Biologic

Once the reference biologic has been subjected to the stress conditions, the resulting molecules are interrogated to determine the quality of reference biologic based on its structural integrity and biological function. Any alternations in protein structures via chemical degradation pathways such as oxidation, deamidation, or isomerization etc. can be measured by LC-MS/MS according to the methods described for structural characterization of the biologics. Similarly, any changes in function can be assessed using techniques known and used in the art.

### (a) Structure

The reference biologic can be analyzed following exposure to one or more of the stress conditions discussed above to identify those structures or substructures within the reference biologic susceptible to modification or degradation. Various characteristics of the as-stressed reference biologic can be analyzed by mass spectrometric analysis to determine any changes or modifications in biologics after exposure to stress conditions. By way of example, the biologic is exposed to one, two or more different temperatures for predetermined periods of time. After the predetermined period of time, the structure of the biologic is interrogated by one or more of the analytic techniques discussed above (for example, the sample preparation and/or the mass spectrometry techniques discussed above), which were used to determine the structure of the reference biologic. Any differences (or the converse, no differences) to the structure of the reference biologic following exposure to the stress condition of interest is noted. The method can be repeated using one or more of the other stress conditions.

Aggregation, which can be caused by chemical or physical perturbations, is a major concern for therapeutic biologics because it can lead to adverse events, immunogenicity or other safety issues. However, aggregation itself is not easily detected by mass spectrometry due to limitations in sensitivity and mass range of mass spectrometers. As a result, other techniques can be used to determine protein aggregates.

In particular, protein aggregates can be measured using various separation or analytical tools (Engelsman et al., PHARM. RES. (2011) 28: 920-933). Conventional separation methods include gel electrophoresis, size exclusion chromatography (SEC), capillary electrophoresis, analytical ultracentrifugation (AUC), and field flow fractionation (FFF), etc. Conventional analytical techniques include dynamic light scattering (DLS), light obscuration (LO), multi-angle light laser scattering (MALLS), UV spectroscopy, light obscuration, micro-flow imaging (MFI), membrane microscopy, and nanoparticle tracking analysis (NTA), etc. The selection of the appropriate method depends upon the forms of aggregates such as small (dimer) or large (polymer), soluble (reversible) or insoluble (irreversible) that are created by each reference biologic. However each method has its own advantages and disadvantages. For example, SEC can quantitatively measure protein aggregates, however, it may not be able to detect soluble aggregates (Arakawa (2007) supra). Light obscuration (LO) can directly measure suspended particles such as insoluble protein aggregates. Nevertheless, it cannot distinguish solid particles from gas-type particles such as air bubbles in solution. Therefore, a “multi-tiered” approach is recommended for protein aggregation analysis. For example, gel electrophoresis (native gel) can be used to verify the presence of protein aggregates. Once observed in the gel, SEC, LO, DLS, AUC and/or FFF can be used as supportive tools to quantitatively measure protein aggregation.

In addition, biologics can also break down into fragments under stress conditions. For example, a protein with quaternary structure can create peptide fragments by mechanisms other than by enzymatic or chemical digestion. Rather, stress induced fragmentation can occur by hydrolysis of amide bonds in peptide backbones. For example, Asp-Gly and Asp-Pro peptide bonds are particularly susceptible to hydrolysis. The stress induced fragments can be determined using, for example, gel electrophoresis together with LC-MS/MS analysis. By way of example, two samples are created for stress testing: a control sample for exposure to ambient conditions and a stressed sample for exposure to stress conditions. To detect stress induced fragmentation, control and stressed samples are subjected to gel electrophoresis under non-reducing conditions. If fragmentation occurs under stress conditions, lower molecular weight fragments are observed following gel electrophoresis which are not observed in the control sample. The gel pieces corresponding to lower molecular weight fragments in the stressed sample and the intact protein in the control sample are excised from the gel. Following in-gel digestion with enzyme(s), the fragmented protein degradation products are identified by LC-MS/MS using CID and/or ETD.

Collectively, the information resulting from the stress tests provides insights into the structure or the substructure of the reference biologic that are affected under each of the conditions. The resulting structural information can then correlated with biological function (discussed below) to determine what stress conditions affect the safety, purity, potency and/or efficacy of the reference biologic.

### (b) Function

In addition to measuring the effect of each stress test has on structure of the biologic, it is important to determine if the structural changes have any effect on safety, efficacy and potency. The changes may be profound or may have little or no effect. As a result, the biological information can further define those aspects of the structure of the biologic that are critical to safety, purity, potency and/or efficacy.

The effect of each stress test can be determined by harvesting the products of each stress test and then running standard biological assays used to determine the biological function of the reference biologic. For example, the activity, in particular the pharmacological activity, of the biologic can be evaluated by in vitro and/or in vivo functional assays. The assays may include, but are not limited to, bioassays, biological assays, binding assays, and enzyme kinetic assays. The particular assays, however, will depend upon the actual biologic being tested. For example, in the case of antibodies, the ability to bind to a target ligand, as well as the binding affinity are important biological assays. Those assays can be used with the as-stressed molecules to determine what effect, if any, those stress conditions have on the biological activity of the protein.

Potency is another important criteria, and represents a measure of relevant biologic function coupled to therapeutic activity of the drug products. Potency tests along with stability and comparability testing often are performed to assure structural attributes associated with product quality (“Guidance for Industry. Potency Tests for Cellular and Gene Therapy Products” U.S. Department of Health and Human Services Food and Drug Administration) (2011). For example, N-terminal fragmentation is produced after a protein exposure under an accelerated stability testing. By way of example, the control and stressed samples are subjected to gel electrophoresis. Assuming that an N-terminal fragment is observed in the gel in the sample resulting from the accelerated aging stress test as compared to the control sample, LC-MS/MS structural characterization can be performed following in-gel digestion of accelerated stability and control samples to determine the N-terminal fragment. After confirming the structural changes resulting from the accelerated stability sample, potency testing using an appropriate bioassay can be carried out to measure the therapeutic activity (i.e., strength) for accelerated stability and control samples. If no difference is observed between accelerated aging sample and the control sample, it appears that the presence of the N-terminal fragments in a sample of biologic has no impact on efficacy for the biologic. However, it is appreciated that the amount of each degradation product in the biologic would need to be established to meet regulatory requirements. If therapeutic activity of the biologic decreases during the accelerated aging study, a follow-up potency test likely will be required. For example, ion exchange chromatography or size exclusion chromatography can be used to fractionate the N-terminal fragment and other degradation products. The resulting fractions can then be subjected to the potency test and LC-MS/MS structural characterization, respectively, to ensure the change of biologic function (as shown as a decrease in potency) is caused by the N-terminal fragment or other degraded products.

Immunogenicity is a major concern with biologics and relates to the ability of the biologic to provoke an immune response in a recipient following administration of the biologic. Similar to potency, immunogenicity can be difficult to predict if any structural changes occur to active molecule(s) Immunogenicity testing often is combined with stability testing to investigate structural features associated with product safety.

The primary structure (peptide sequence) and the high order structures (secondary, tertiary, and quaternary structures) of therapeutic proteins play important roles in contributing immunogenicity. (“Guidance for Industry Immunogenicity Assessment for Therapeutic Protein Products” U.S. Department of Health and Human Services Food and Drug Administration (February 2013)). For example, after stress testing, a therapeutic protein product usually is subjected to structural characterization by LC-MS/MS against a control sample. Through LC-MS profiling, degradation products (for example, Asn deamidation, Cys oxidation, or different glycoforms) may be observed, which could result in causing protein aggregation or result in changes to the structures of the biologic involved in function. To determine whether these structural modifications result in aggregation or conformational changes, following identification by LC-MS/MS, gel electrophoresis or size exclusion chromatography can be used to detect the presence of protein aggregates. In addition, HDX MS can be used to probe conformational changes that are associated with biological function. For example, if there are differences in the uptake of HD exchange in peptide epitopes between unstressed and stressed biologic samples, these differences are indicative that conformational changes occur in antibody-antigen binding site(s). In other words, there may be an increase in immunogenicity of the biologic. Given that it is difficult to predict immunogenicity of the stress induced protein products, even when aggregates are formed, immunogenicity testing assays in animal models and/or via clinical studies can be necessary to assure the safety of the biologic.

### (III) Creation of Map of Critical Attributes and its Applications

Once the structural and function features of the reference biologic have been determined before and after stress testing the resulting data can be analyzed to determine which physical and/or chemical conditions, if any, materially affect the structure and/or function of the reference biologic. The data preferably is analyzed computationally using a conventional computer or computer system to identify such conditions, and to identify critical structure or substructures of the biologic. The data can also be analyzed, preferably computationally, to identify operational parameters used in the expression, purification, formulation, and storage of the reference biologic that result in or pose a risk of degrading, modifying, or contaminating the biological drug.

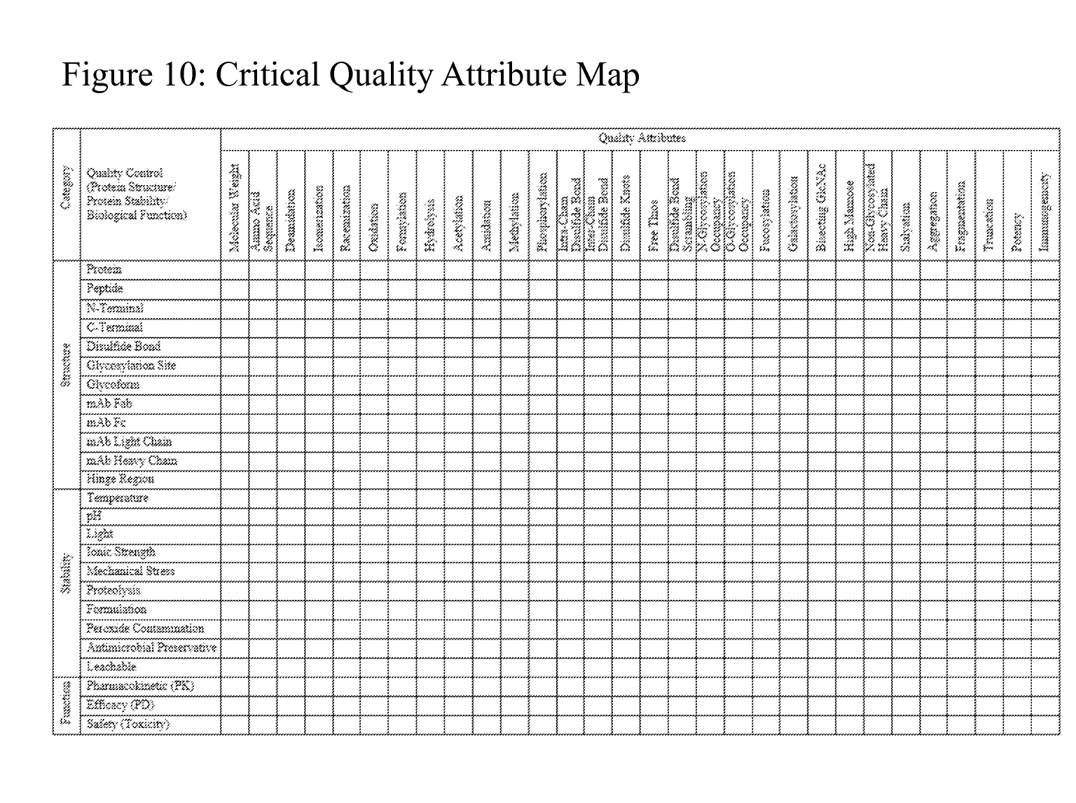

This information can then be assimilated to produce a map or fingerprint (see, e.g., FIG. 10) that identifies portions of the biologic that are at risk of modification, degradation or aggregation during its lifetime (for example, during manufacture, formulation, storage or use), which may affect the function of the biologic, and/or which may have limited or no effect on the function of the biologic. The map comprises a listing of a set of reproducible analytical techniques and their outputs which uniquely characterize the molecule, and thus enables determination of (a) which attributes actually relate to and/or impart function, or not, and (b) which processing parameters degrade or risk degrading the structural features of the biologic known to be material to its function and safety.

The map may be exploited during development of expression and purification protocols conforming to GMP. It predicts triggers of, and development solutions to, degradation, aggregation, and aberrant modification. It can function as a quality control tool in commercial scale production of the biologic so as to ensure reliable manufacture of consistent quality product falling within realistic specifications guaranteeing its safety, purity, potency and/or efficacy throughout its lifetime. When, for whatever reason, a process step is changed before or after approval, the map enables comparability testing. It also serves as a specification or benchmark for long term stability testing, and importantly can serve as a roadmap for specific development and regulatory process designs and decisions, including tailored formulation to mitigate or enhance the identified key fine structures of the molecule.

The map can also be used as a standard in the development of biosimilars. In particular, the map can be used in the definition and/or generation of biosimilars. For example, the map may be used (a) as a target to design the biosimilar development and regulatory processes, and (b) as a qualification of biosimilarity for a given protein or batch or proteins as related to a previously marketed protein approved by a regulatory agency. Once a map for a reference biologic has been obtained, the map can be used to determine whether another biologic, for example, made under different conditions (for example, expressed using a different expression vector, expression host, culture conditions, purification conditions, and formulation conditions) is biosimilar to the reference biologic. The method comprises analyzing a batch of material assure that its attributes satisfy a critical features identified in the map. Once satisfied, the batch of material can then be marketed as an approved drug.

### EXAMPLES

The following Examples are merely illustrative and are not intended to limit the scope or content of the invention in any way.

### Example 1

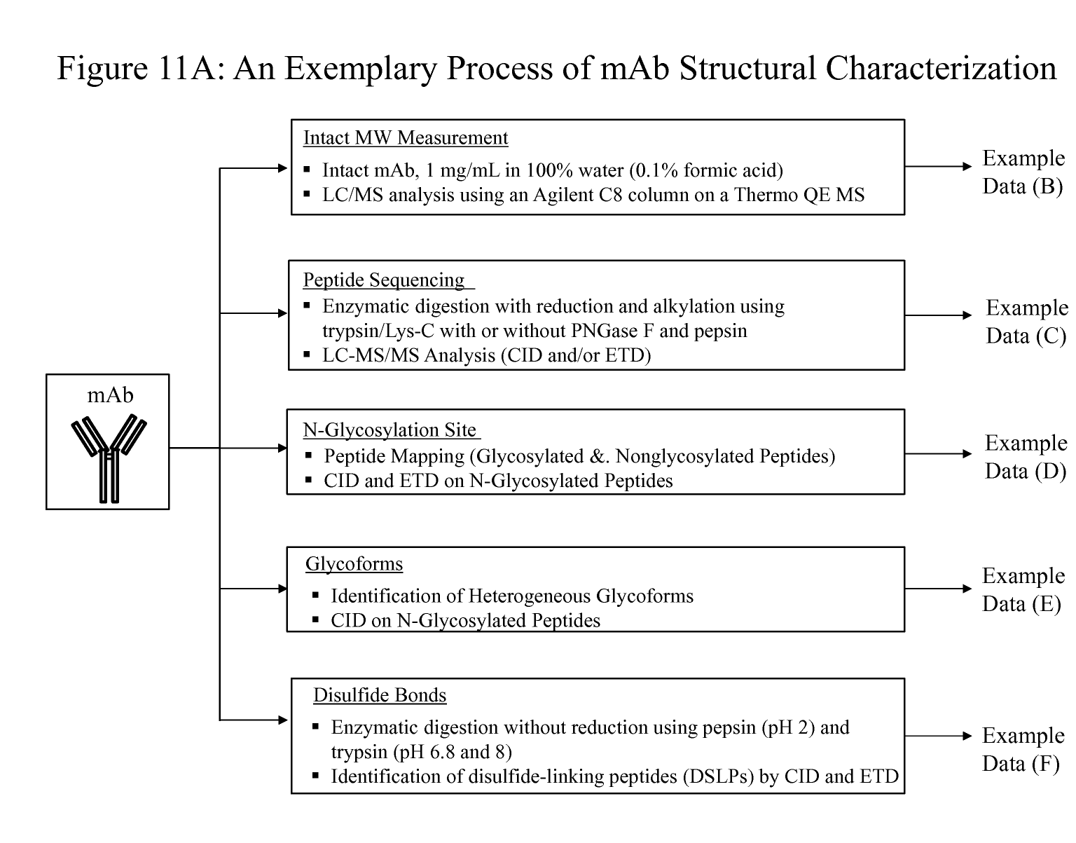

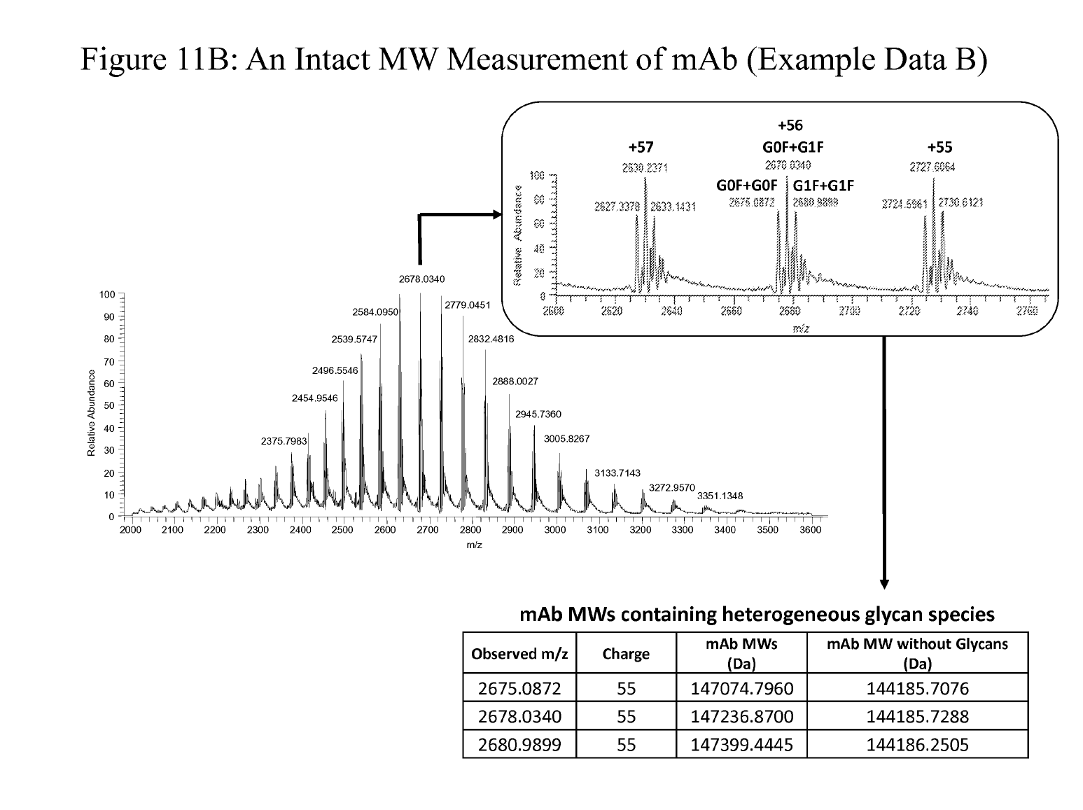

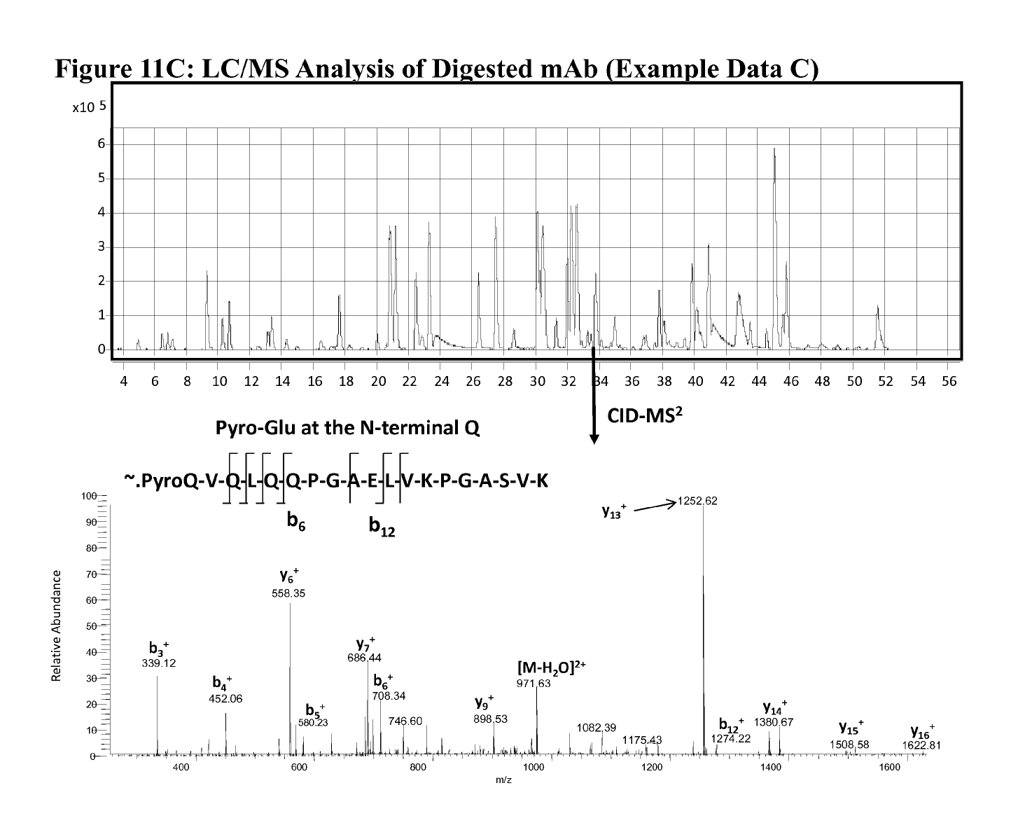

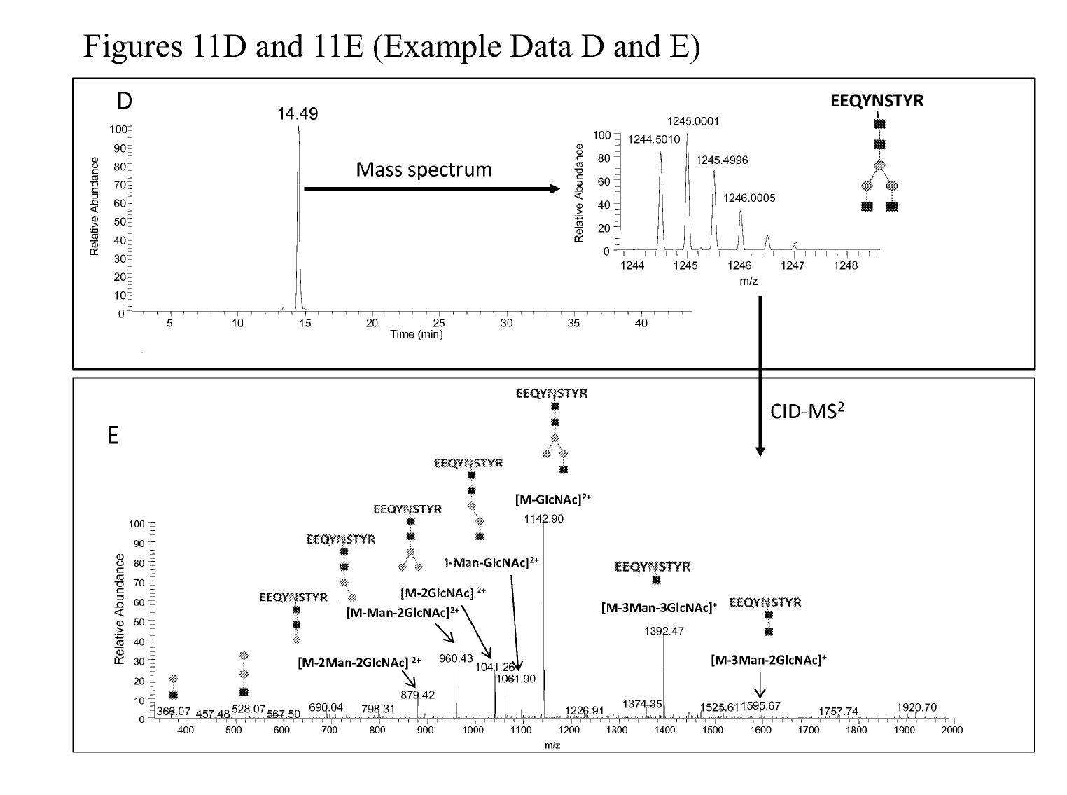

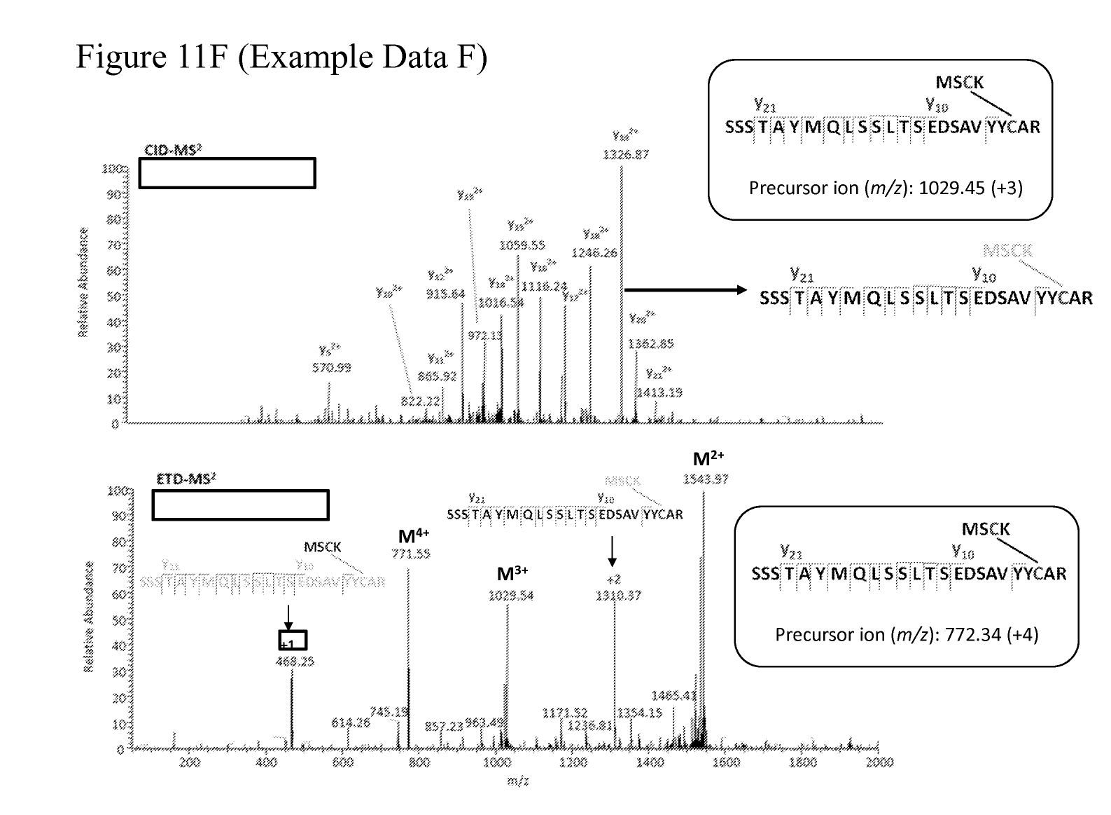

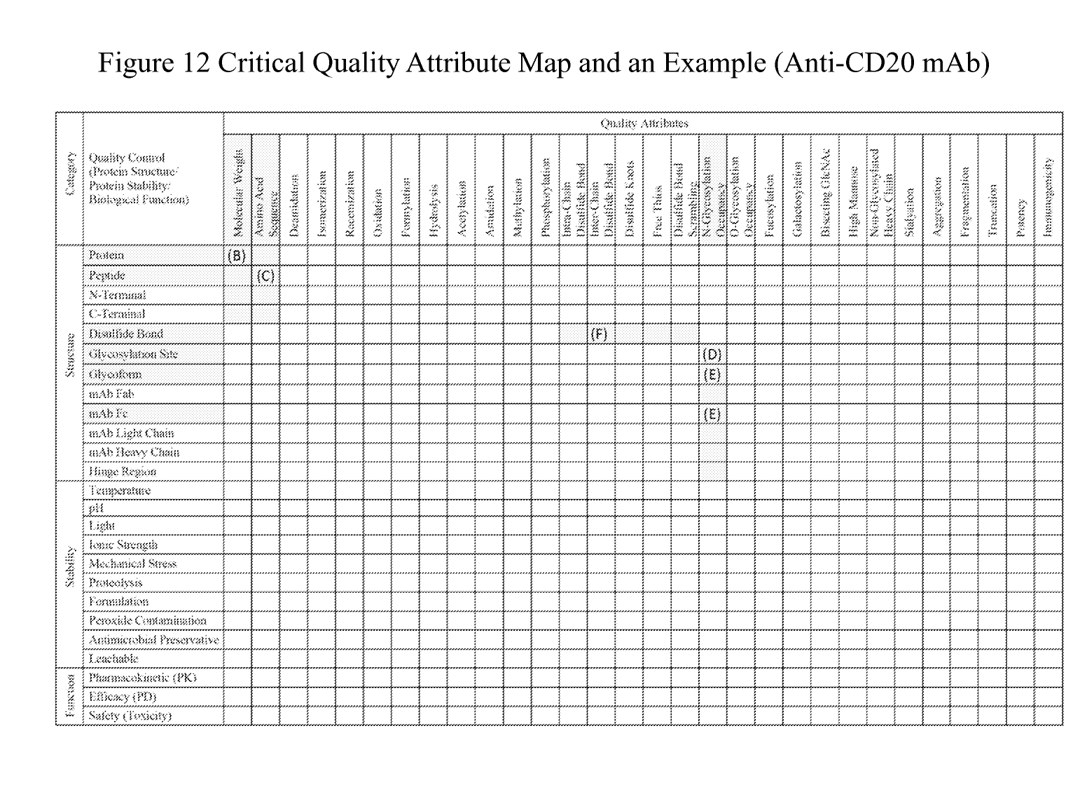

An exemplary anti-CD20 monoclonal antibody (mAb) is used in this example to demonstrate the use of certain analytical techniques described above to obtain structural information about the biologic (FIG. 11A). The anti-CD20 mAb (1 mg/mL) is subjected to enzymatic digestion using a single enzyme and multiple enzymes with and without reduction. The intact molecular weight of the anti-CD20 mAb is determined by LC/MS (FIG. 11B) using an Agilent C8 column on a Thermo QE mass spectrometer. To ensure coverage of the full sequence of the anti-CD20 antibody, a single enzyme (for example, pepsin, trypsin) and various combinations of multiple enzymes (for example, trypsin/Lys-C, trypsin/PNGase F) are used to yield peptide fragments which are subsequently analyzed by LC-MS/MS using CID and ETD to reveal the molecular weight of the antibody or the antibody without glycans (FIG. 11C). For the determination of N-glycosylation site(s), digested mAb without PNGase F is used to obtain N-glycosylated peptides and non-glycosylated peptides. The precursor ion of each N-glycosylated peptide is monitored by LC-MS/MS (FIG. 11D) and subsequently analyzed by CID (CID-MS²) and ETD (ETD-MS²). By identifying N-site specific glycoforms using CID (FIG. 11E), the N-glycosylation site is then confirmed. To locate the disulfide bonds on the anti-CD20 antibody, the mAb digest without reduction is used to obtain disulfide-linked peptide fragments, which are then analyzed by CID and ETD (FIG. 11F). Additionally, various mAb digests (pH 2, 6.6, and 8) are monitored to ensure disulfide scrambling is not induced from the sample process. Following establishment of the structure of a biologic reference such as the anti-CD20 mAb, this structural information can be used to identify quality control attributes that can be used to create a CQA map (see FIGS. 11A-11E and FIG. 12) that can be used to monitor production of this biologic.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of generating a map comprising data uniquely characterizing a biological drug exhibiting a desired biologic activity, the method comprising the steps of:
(a) generating through mass spectroscopic analysis data indicative of the structure of an active, reference biologic;
(b) subjecting the reference biologic to one or more stress conditions selected from high temperature, physiological temperature, pH change, light, lyophilization and reconstitution, changes in ionic environment, mechanical stress, and accelerated aging;
(c) generating through mass spectroscopic analysis data indicative of the structure of the as-stressed reference biologic, and optionally derivatives, fragments or degradation products thereof;
(d) computationally analyzing the data generated in step (a) and/or (c) to determine which operational parameters used in the expression, purification, formulation, or storage of said reference biologic result in or pose a risk of degrading, modifying, or contaminating the biological drug; and
(e) preparing a map comprising a record of selected analyses and results thereof informative of the structural aspects of the reference biologic that are critical to its stability and biologic activity using the data generated in step (a) and/or (c), and optionally specifies the conditions of expression, purification, formulation, or storage thereby enhancing the chance of producing safe and efficacious biologic drug meeting said structural aspects that are critical to its stability and biological activity.

2. The method of claim 1 further comprising the step of assaying the as-stressed reference biologic, and optionally derivatives, fragments or degradation products thereof for drug activity before step (e).

3. The method of claim 1, wherein the map additionally specifies the physical or chemical conditions which risk alteration of structural features of the biologic drug critical to its safety, purity, potency or efficacy.

4. The method of claim 1, 2, or 3, wherein the map is of sufficient detail to serve as quality assurance criteria to qualify a batch of the biological drug at some stage of its manufacture as biosimilar to another batch of the biological drug at the same stage produced separately.

5. The method of claim 4, wherein the map is of sufficient detail to serve as criteria to qualify for regulatory purposes one batch of the biological drug as biosimilar to another batch of the biological drug produced separately.

6. The method of claim 4, wherein the map is of sufficient detail to serve as criteria to qualify one batch of the biological drug as biosimilar to another batch of the biological drug produced separately using an altered protocol.

7. The method of claim 4, wherein step (c) comprises determining the mass spectrometry profile in a sample of one or more stressed species selected from the group consisting of derivatized, truncated, oxidized, methylated, deaminated, aggregated, differentially glycosylated, improperly disulfide bonded, or structurally intact protein species, fragments thereof, and contaminants therein.

8. The method of claim 4, wherein the mass spectrometry analysis data generated in step (a) or step (c) is generated by fragmenting or chemically modifying the reference biologic, the as-stressed reference biologic, and optionally derivatives, fragments or degradation products therein before or while subjecting the sample to mass spectrometric analysis.

9. The method of claim 4, wherein the generation of mass spectrometry data is effected through one or more of the techniques selected from the group consisting of electron transfer dissociation mass spectrometry, collision induced dissociation mass spectrometry, higher-energy collisional dissociation mass spectrometry, electron capture dissociation mass spectrometry, infrared multi-photon dissociation mass spectrometry, hydrogen/deuterium exchange mass spectrometry, MS², MS³, and LC-MS.

10. The method of claim 4, wherein mass spectrometry analysis data in steps (a) or (c) also are generated by analytical techniques selected from the group consisting of electrophoresis, selective proteolysis, UV spectra analysis, IR spectra analysis and MRI spectra analysis.

11. A method of qualifying a given batch of biologic as biosimilar to a previously marketed biologic approved by a regulatory agency, the batch having been made by purification of expression products of a host cell in culture, the method comprising analyzing the batch to assure that its attributes satisfy a critical quality attribute map produced in accordance with the method of claim 4.

12. The method of claim 11 comprising the additional step of marketing the batch as an approved drug.

13. The method of claim 11 comprising the additional step of marketing another batch produced using the same protocol used to produce said given batch as an approved drug.

14. The method of any one of claims 1-13, wherein the reference biologic is representative of the biological drug.

15. The method of any one of claims 1-13, wherein the biologic or biological drug is a protein or peptide.

16. A method of generating a map comprising data uniquely characterizing a biological drug exhibiting a desired biologic activity, the method comprising the steps of:
(a) generating through mass spectroscopic analysis data indicative of the structure of an active, reference biologic;
(b) stressing the reference biologic by subjecting the reference biologic to in vivo conditions;
(c) generating through mass spectroscopic analysis data indicative of the structure of the as-stressed reference biologic, and optionally derivatives, fragments or degradation products thereof;
(d) computationally analyzing the data generated in step (a) and/or (c) to determine which structural aspects of the reference biologic are critical to its stability and biological activity under in vivo conditions; and
(e) preparing a map comprising a record of selected analyses and results thereof informative of the structural aspects of the reference biologic that are critical to its stability and biologic activity using the data generated in step (a) and/or (c).

17. The method of claim 16, further comprising the step of assaying the as-stressed reference biologic, and optionally derivatives, fragments or degradation products thereof for drug activity before step (e).

18. The method of claim 16, wherein in step (b), the reference biologic is exposed to physiological temperature or proteases.

19. The method of claim 16, 17, or 18, wherein the map is of sufficient detail to serve as quality assurance criteria to qualify a batch of the biological drug as biosimilar to another batch of the biological drug produced separately.

20. The method of claim 19, wherein the map is of sufficient detail to serve as criteria to qualify for regulatory purposes one batch of the biological drug as biosimilar to another batch of the biological drug produced separately.

21. The method of claim 19, wherein the generation of mass spectrometry data is effected through one or more of the techniques selected from the group consisting of electron transfer dissociation mass spectrometry, collision induced dissociation mass spectrometry, higher-energy collisional dissociation mass spectrometry, electron capture dissociation mass spectrometry, infrared multi-photon dissociation mass spectrometry, hydrogen/deuterium exchange mass spectrometry, MS², MS³, and LC-MS.

22. The method of any one of claims 16-21, wherein the biologic or biological drug is a protein or peptide.
